(19) United States
(12) Patent Application Publication
(10) Pub. No.: US 2011/0060053 A1
(43) Pub. Date: Mar. 10, 2011

(54) Crystalline Modifications of 6-Dimethylaminomethyl-1-(3-methoxyphenyl)-cyclohexane-1,3-diol

(75) Inventors: Michael Gruss, Aachen (DE); Helmut Heinrich Buschmann, Aachen (DE)

(73) Assignee: Gruenenthal GmbH, Aachen (DE)

(21) Appl. No.: 12/880,614

(22) Filed: Sep. 13, 2010

Related U.S. Application Data
(60) Provisional application No. 61/242,083, filed on Sep. 14, 2009.

(30) Foreign Application Priority Data
Sep. 9, 2002 (EP) 09011721

(51) Int. Cl. A61K 31/135; C07C 217/74; A61P 29/00

(52) U.S. Cl. 514/646; 564/443

(57) ABSTRACT

Crystalline modifications of (1R,3R,6R)-6-dimethylaminomethyl-1-(3-methoxyphenyl)-cyclohexane-1,3-diol, (1S,3S,6S)-6-dimethylaminomethyl-1-(3-methoxyphenyl)-cyclohexane-1,3-diol and mixtures thereof, pharmaceutical compositions and medicaments comprising these modifications, the use of these modifications as well as to a process for the enrichment of (1R,3R,6R)- or (1S,3S,6S)-6-dimethylaminomethyl-1-(3-methoxyphenyl)-cyclohexane-1,3-diol.

### BACKGROUND OF THE INVENTION

The present invention relates to crystalline modifications of (1R,3R,6R)-6-dimethylaminomethyl-1-(3-methoxyphenyl)-cyclohexane-1,3-diol, (1S,3S,6S)-6-dimethylaminomethyl-1-(3-methoxyphenyl)-cyclohexane-1,3-diol and mixtures thereof, pharmaceutical compositions and medicaments comprising these modifications, the use of these modifications as well as to a process for the enrichment of (1R,3R,6R)- or (1S,3S,6S)-6-dimethylaminomethyl-1-(3-methoxyphenyl)-cyclohexane-1,3-diol.

(1RS,3RS,6RS)-6-dimethylaminomethyl-1-(3-methoxyphenyl)-cyclohexane-1,3-diol—also known as Axomadol (e.g. WHO Drug Information, Vol. 16, No. 2, 2002, List 87)—is a synthetic, centrally active analgesic that is suitable for the treatment of moderate to severe, acute or chronic pain. Axomadol and methods for its production are known from US RE37,355 E.

### SUMMARY OF THE INVENTION

One object of the present invention was to make the individual enantiomers of Axomadol, i.e. (1R,3R,6R)-6-dimethylaminomethyl-1-(3-methoxyphenyl)-cyclohexane-1,3-diol and (1S,3S,6S)-6-dimethylaminomethyl-1-(3-methoxyphenyl)-cyclohexane-1,3-diol and thus also (1RS,3RS,6RS)-6-dimethylaminomethyl-1-(3-methoxyphenyl)-cyclohexane-1,3-diol accessible per se, i.e. in the form of the free base, with high yields and high purity.

This and other objects have been achieved by the invention as described hereinafter.

It has surprisingly been found that under suitable conditions (1R,3R,6R)-6-dimethylaminomethyl-1-(3-methoxyphenyl)-cyclohexane-1,3-diol and (1S,3S,6S)-6-dimethylaminomethyl-1-(3-methoxyphenyl)-cyclohexane-1,3-diol can be obtained in crystalline form, in particular in form of polymorphs A, B and C as described hereinafter.

These crystalline forms make it possible to obtain (1R,3R,6R)-6-dimethylaminomethyl-1-(3-methoxyphenyl)-cyclohexane-1,3-diol and (1S,3S,6S)-6-dimethylaminomethyl-1-(3-methoxyphenyl)-cyclohexane-1,3-diol and therefore also mixtures thereof, in particular racemic (1RS,3RS,6RS)-6-dimethylaminomethyl-1-(3-methoxyphenyl)-cyclohexane-1,3-diol in the form of the free base, with high yields and high purity. These forms are further distinguished in that they are very easy to handle and allow an exact metering of the active ingredient.

Moreover, different polymorphs of (1R,3R,6R)-6-dimethylaminomethyl-1-(3-methoxyphenyl)-cyclohexane-1,3-diol and (1S,3S,6S)-6-dimethylaminomethyl-1-(3-methoxyphenyl)-cyclohexane-1,3-diol have fundamentally different properties, which may provide further advantages.

On the one hand, the advantages may be based on a particular physical property of a particular modification, for example in relation to the handling or storage thereof, for example thermodynamic stability; crystal morphology, in particular structure, size, colour; density; bulk density; hardness; deformability; calorimetric characteristics, in particular melting point; solubility properties, in particular intrinsic rate of dissolution and equilibrium solubility; hygroscopicity; relative moisture profile; adhesion etc.

On the other hand, the crystalline modifications may also have improved chemical properties. For example, it is known that a lower hygroscopicity can lead to improved chemical stability and longer storage lives for chemical compounds.

One aspect of the present invention relates to a crystalline modification of (1R,3R,6R)-6-dimethylaminomethyl-1-(3-methoxyphenyl)-cyclohexane-1,3-diol, or (1S,3S,6S)-6-dimethylaminomethyl-1-(3-methoxyphenyl)-cyclohexane-1,3-diol, or a mixture thereof.

If the enantiomers are present in a mixture such a mixture may contain the enantiomers in racemic or non-racemic amounts. A non-racemic amount can contain the enantiomers in any possible ratio, for example, in a ratio of 60±5:40±5; 70±5:30±5; 80±5:20±5 or 90±5:10±5.

### BRIEF DESCRIPTION OF THE DRAWINGS

The invention will be described in further detail hereinafter with reference to the accompanying drawing figures, in which:

FIG. 1 shows a calculated diffractogram of crystalline modification A;

FIG. 2 shows a measured diffractogram of crystalline modification A;

FIG. 3 shows a comparison of the calculated diffractogram according to FIG. 1 and the measured diffractogram of crystalline modification A according to FIG. 2;

FIG. 4 shows a Raman spectrum of crystalline modification A;

FIG. 5 shows a calculated diffractogram of crystalline modification B; and

FIG. 6 shows a measured diffractogram of crystalline modification C.

### DETAILED DESCRIPTION

A further aspect of the present invention relates to a crystalline modification A (polymorph A). The crystalline modification A has an X-ray diffraction peak at 10.69±0.20 (2Θ).

Preferably, the crystalline modification A according to the present invention may additionally have at least one X-ray diffraction peak selected from the group consisting of 12.81±0.20 (2Θ), 13.82±0.20 (2Θ), 13.88±0.20 (2Θ), 16.71±0.20 (2Θ), 18.31±0.20 (2Θ), 18.76±0.20 (2Θ), 19.52±0.20 (2Θ), 20.56±0.20 (2Θ), 20.60±0.20 (2Θ), 20.61±0.20 (2Θ), 21.42±0.20 (2Θ), 22.63±0.20 (2Θ), 23.85±0.20 (2Θ) and 26.34±0.20 (2Θ).

The crystalline modification A according to the present invention may further be characterized in that as well as the X-ray diffraction peak at 10.69±0.20 (2Θ) and optionally one or more X-ray diffraction peaks selected from the group consisting of 12.81±0.20 (2Θ), 13.82±0.20 (2Θ), 13.88±0.20 (2Θ), 16.71±0.20 (2Θ), 18.31±0.20 (2Θ), 18.76±0.20 (2Θ), 19.52±0.20 (2Θ), 20.56±0.20 (2Θ), 20.60±0.20 (2Θ), 20.61±0.20 (2Θ), 21.42±0.20 (2Θ), 22.63±0.20 (2Θ), 23.85±0.20 (2Θ) and 26.34±0.20 (2Θ), it additionally has at least one X-ray diffraction peak selected from the group consisting of 18.32±0.20 (2Θ), 24.79±0.20 (2Θ), 25.08±0.20 (2Θ), 28.66±0.20 (2Θ), 30.33±0.20 (2Θ), 33.05±0.20 (2Θ) and 38.36±0.20 (2Θ).

Furthermore, the crystalline modification A according to the present invention may be characterized in that as well as the X-ray diffraction peak at 10.69±0.20 (2Θ) and optionally one or more X-ray diffraction peaks selected from the group consisting of 12.81±0.20 (2Θ), 13.82±0.20 (2Θ), 13.88±0.20 (2Θ), 16.71±0.20 (2Θ), 18.31±0.20 (2Θ), 18.76±0.20 (2Θ), 19.52±0.20 (2Θ), 20.56±0.20 (2Θ), 20.60±0.20 (2Θ), 20.61±0.20 (2Θ), 21.42±0.20 (2Θ), 22.63±0.20 (2Θ), 23.85±0.20 (2Θ) and 26.34±0.20 (2Θ) and optionally one or more X-ray diffraction peaks selected from the group consisting of 18.32±0.20 (2Θ), 24.79±0.20 (2Θ), 25.08±0.20 (2Θ), 28.66±0.20 (2Θ), 30.33±0.20 (2Θ), 33.05±0.20 (2Θ) and 38.36±0.20 (2Θ), it additionally has at least one X-ray diffraction peak selected from the group consisting of 10.24±0.20 (2Θ), 10.77±0.20 (2Θ), 14.22±0.20 (2Θ), 17.52±0.20 (2Θ), 19.89±0.20 (2Θ), 21.48±0.20 (2Θ), 21.64±0.20 (2Θ), 23.22±0.20 (2Θ), 23.37±0.20 (2Θ), 25.67±0.20 (2Θ), 25.77±0.20 (2Θ), 26.33±0.20 (2Θ), 27.85±0.20 (2Θ), 28.59±0.20 (2Θ), 28.82±0.20 (2Θ), 29.43±0.20 (2Θ), 29.67±0.20 (2Θ), 29.93±0.20 (2Θ), 30.11±0.20 (2Θ), 30.17±0.20 (2Θ), 30.52±0.20 (2Θ), 31.62±0.20 (2Θ), 32.31±0.20 (2Θ), 32.46±0.20 (2Θ), 32.59±0.20 (2Θ), 32.71±0.20 (2Θ), 33.67±0.20 (2Θ), 33.71±0.20 (2Θ), 33.79±0.20 (2Θ), 33.92±0.20 (2Θ), 33.92±0.20 (2Θ), 34.23±0.20 (2Θ), 34.28±0.20 (2Θ), 34.39±0.20 (2Θ), 35.02±0.20 (2Θ), 35.24±0.20 (2Θ), 35.46±0.20 (2Θ), 35.61±0.20 (2Θ), 35.80±0.20 (2Θ), 36.53±0.20 (2Θ), 36.75±0.20 (2Θ), 36.92±0.20 (2Θ), 37.14±0.20 (2Θ), 37.16±0.20 (2Θ), 37.34±0.20 (2Θ), 38.08±0.20 (2Θ), 38.38±0.20 (2Θ), 38.45±0.20 (2Θ), 38.82±0.20 (2Θ), 39.29±0.20 (2Θ), 39.36±0.20 (2Θ), 39.47±0.20 (2Θ) and 39.63±0.20 (2Θ).

The crystalline modification A according to the invention may also be characterized in that as well as the X-ray diffraction peak at 10.69±0.20 (2Θ) and optionally one or more X-ray diffraction peaks selected from the group consisting of 12.81±0.20 (2Θ), 13.82±0.20 (2Θ), 13.88±0.20 (2Θ), 16.71±0.20 (2Θ), 18.31±0.20 (2Θ), 18.76±0.20 (2Θ), 19.52±0.20 (2Θ), 20.56±0.20 (2Θ), 20.60±0.20 (2Θ), 20.61±0.20 (2Θ), 21.42±0.20 (2Θ), 22.63±0.20 (2Θ), 23.85±0.20 (2Θ) and 26.34±0.20 (2Θ) and optionally one or more X-ray diffraction peaks selected from the group consisting of 18.32±0.20 (2Θ), 24.79±0.20 (2Θ), 25.08±0.20 (2Θ), 28.66±0.20 (2Θ), 30.33±0.20 (2Θ), 33.05±0.20 (2Θ) and 38.36±0.20 (2Θ), and optionally one or more X-ray diffraction peaks selected from the group consisting of 10.24±0.20 (2Θ), 10.77±0.20 (2Θ), 14.22±0.20 (2Θ), 17.52±0.20 (2Θ), 19.89±0.20 (2Θ), 21.48±0.20 (2Θ), 21.64±0.20 (2Θ), 23.22±0.20 (2Θ), 23.37±0.20 (2Θ), 25.67±0.20 (2Θ), 25.77±0.20 (2Θ), 26.33±0.20 (2Θ), 27.85±0.20 (2Θ), 28.59±0.20 (2Θ), 28.82±0.20 (2Θ), 29.43±0.20 (2Θ), 29.67±0.20 (2Θ), 29.93±0.20 (2Θ), 30.11±0.20 (2Θ), 30.17±0.20 (2Θ), 30.52±0.20 (2Θ), 31.62±0.20 (2Θ), 32.31±0.20 (2Θ), 32.46±0.20 (2Θ), 32.59±0.20 (2Θ), 32.71±0.20 (2Θ), 33.67±0.20 (2Θ), 33.71±0.20 (2Θ), 33.79±0.20 (2Θ), 33.92±0.20 (2Θ), 33.92±0.20 (2Θ), 34.23±0.20 (2Θ), 34.28±0.20 (2Θ), 34.39±0.20 (2Θ), 35.02±0.20 (2Θ), 35.24±0.20 (2Θ), 35.46±0.20 (2Θ), 35.61±0.20 (2Θ), 35.80±0.20 (2Θ), 36.53±0.20 (2Θ), 36.75±0.20 (2Θ), 36.92±0.20 (2Θ), 37.14±0.20 (2Θ), 37.16±0.20 (2Θ), 37.34±0.20 (2Θ), 38.08±0.20 (2Θ), 38.38±0.20 (2Θ), 38.45±0.20 (2Θ), 38.82±0.20 (2Θ), 39.29±0.20 (2Θ), 39.36±0.20 (2Θ), 39.47±0.20 (2Θ) and 39.63±0.20 (2Θ), it additionally has at least one X-ray diffraction peak selected from the group consisting of 18.62±0.20 (2Θ), 20.73±0.20 (2Θ), 23.47±0.20 (2Θ), 26.96±0.20 (2Θ), 27.97±0.20 (2Θ), 28.07±0.20 (2Θ), 28.37±0.20 (2Θ), 28.56±0.20 (2Θ), 28.57±0.20 (2Θ), 30.02±0.20 (2Θ), 31.06±0.20 (2Θ), 32.51±0.20 (2Θ), 33.03±0.20 (2Θ), 33.48±0.20 (2Θ), 35,89±0.20 (2Θ), 37.11±0.20 (2Θ), 37.62±0.20 (2Θ), 39.11±0.20 (2Θ), 39.28±0.20 (2Θ), 39.48±0.20 (2Θ) and 39.50±0.20 (2Θ).

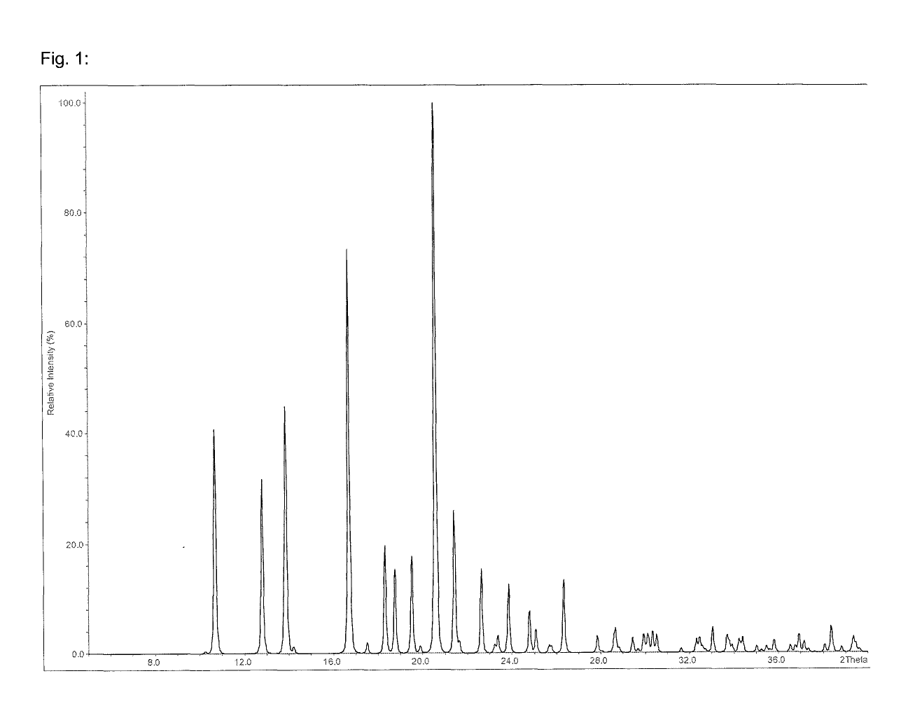

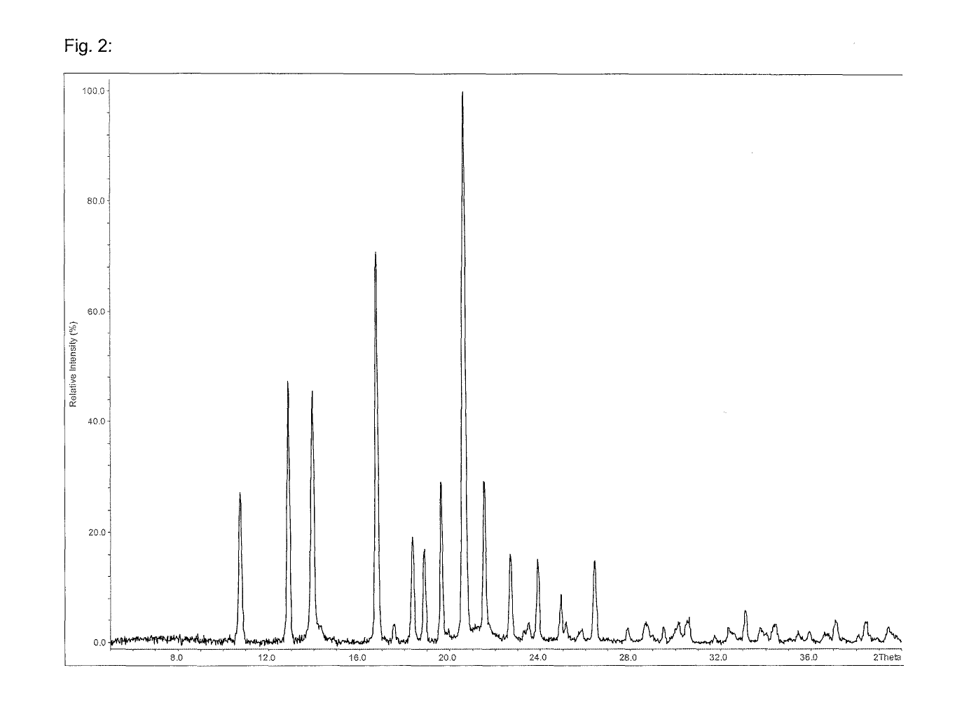

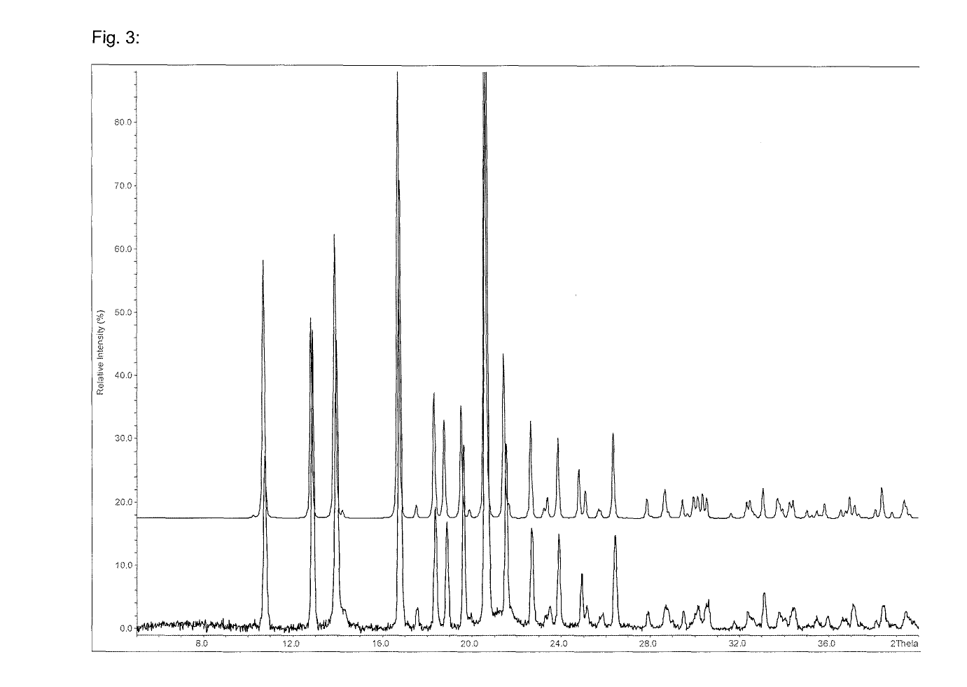

FIG. 1 shows a calculated x-ray powder diffractogram of crystalline modification A. FIG. 2 shows a measured x-ray powder diffractogram of crystalline modification A. FIG. 3 shows a comparison of the calculated x-ray powder diffractogram according to FIG. 1 and the measured x-ray powder diffractogram according to FIG. 2 of crystalline modification A.

In DSC analyses, the crystalline modification A according to the present invention preferably exhibits an endothermal event with a peak temperature at 113-121° C., more preferably at 114-120° C., even more preferably at 115-119° C. and in particular at 115-118° C.

The crystalline form A according to the present invention may further be characterized in that it has at least a Raman band at 993±4 cm⁻¹ (VS) and/or a Raman band at 241±4 cm⁻¹ (M).

The crystalline form A according to the present invention may further be characterized in that it has a Raman band at 993±4 cm⁻¹ (VS) and/or a Raman band at 241±4 cm⁻¹ (M) and/or one or more Raman bands selected from the group consisting of 200±4 cm⁻¹ (W), 279±4 cm⁻¹ (W), 633±4 cm⁻¹ (W), 701±4 cm⁻¹ (W), 726±4 cm⁻¹ (W), 843±4 cm⁻¹ (W), 855±4 cm⁻¹ (W), 1065±4 cm⁻¹ (W), 1082±4 cm⁻¹ (W), 1194±4 cm⁻¹ (W), 1258±4 cm⁻¹ (W), 1431±4 cm⁻¹ (W), 1445±4 cm⁻¹ (W), 1458±4 cm⁻¹ (W), 1468±4 cm⁻¹ (W), 1596±4 cm⁻¹ (W), 1699±4 cm⁻¹ (W), 2786±4 cm⁻¹ (W), 2834±4 cm⁻¹ (W), 2875±4 cm⁻¹ (W), 2928±4 cm⁻¹ (W), 2943±4 cm⁻¹ (W) and 3062±4 cm⁻¹ (W) and/or one or more Raman bands selected from the group consisting of 148±4 cm⁻¹ (VW), 338±4 cm⁻¹ (VW), 365±4 cm⁻¹ (VW), 400±4 cm⁻¹ (VW), 428±4 cm⁻¹ (VW), 486±4 cm⁻¹ (VW), 537±4 cm⁻¹ (VW), 555±4 cm⁻¹ (VW), 621±4 cm⁻¹ (VW), 796±4 cm⁻¹ (VW), 869±4 cm⁻¹ (VW), 885±4 cm⁻¹ (VW), 925±4 cm⁻¹ (VW), 956±4 cm⁻¹ (VW), 970±4 cm⁻¹ (VW), 1011±4 cm⁻¹ (VW), 1038±4 cm⁻¹ (VW), 1091±4 cm⁻¹ (VW), 1096±4 cm⁻¹ (VW), 1109±4 cm⁻¹ (VW), 1156±4 cm⁻¹ (VW), 1171±4 cm⁻¹ (VW), 1218±4 cm⁻¹ (VW), 1282±4 cm⁻¹ (VW), 1305±4 cm⁻¹ (VW), 1323±4 cm⁻¹ (VW), 1343±4 cm⁻¹ (VW), 1355±4 cm⁻¹ (VW), 1385±4 cm⁻¹ (VW), 1407±4 cm⁻¹ (VW), 1422±4 cm⁻¹ (VW), 1653±4 cm⁻¹ (VW), 1759±4 cm⁻¹ (VW), 1769±4 cm⁻¹ (VW), 2899±4 cm⁻¹ (VW), 2953±4 cm⁻¹ (VVV), 2970±4 cm⁻¹ (VW), 2993±4 cm⁻¹ (VW), 3011±4 cm⁻¹ (VW), and 3087±4 cm⁻¹ (VW).

The crystalline form A according to the present invention may further be characterized in that it has one or more Raman bands selected from the group consisting of 148±4 cm⁻¹ (VW), 200±4 cm⁻¹ (W), 241±4 cm⁻¹ (M), 279±4 cm⁻¹ (W), 338±4 cm⁻¹ (VW), 365±4 cm⁻¹ (VW), 400±4 cm⁻¹ (VW), 428±4 cm⁻¹ (VW), 452±4 cm⁻¹ (W), 486±4 cm⁻¹ (VW), 537±4 cm⁻¹ (VW), 555±4 cm⁻¹ (VW), 621±4 cm⁻¹ (VW), 633±4 cm⁻¹ (W), 701±4 cm⁻¹ (W), 726±4 cm⁻¹ (W), 796±4 cm⁻¹ (VW), 843±4 cm⁻¹ (W), 855±4 cm⁻¹ (W), 869±4 cm⁻¹ (VW), 885±4 cm⁻¹ (VW), 925±4 cm⁻¹ (VW), 956±4 cm⁻¹ (VW), 970±4 cm⁻¹ (VW), 993±4 cm⁻¹ (VS), 1011±4 cm⁻¹ (VW), 1038±4 cm⁻¹ (VW), 1065±4 cm⁻¹ (W), 1082±4 cm⁻¹ (W), 1091±4 cm⁻¹ (VW), 1096±4 cm⁻¹ (VW), 1109±4 cm⁻¹ (VW), 1156±4 cm⁻¹ (VW), 1171±4 cm⁻¹ (VW), 1194±4 cm⁻¹ (W), 1218±4 cm⁻¹ (VW), 1258±4 cm⁻¹ (W), 1282±4 cm⁻¹ (VW), 1305±4 cm⁻¹ (VW), 1323±4 cm⁻¹ (VW), 1343±4 cm⁻¹ (VW), 1355±4 cm⁻¹ (VW), 1385±4 cm⁻¹ (VW), 1407±4 cm⁻¹ (VW), 1422±4 cm⁻¹ (VW), 1431±4 cm⁻¹ (W), 1445±4 cm⁻¹ (W), 1458±4 cm⁻¹ (W), 1468±4 cm⁻¹ (W), 1596±4 cm⁻¹ (W), 1653±4 cm⁻¹ (VW), 1699±4 cm⁻¹ (W), 1759±4 cm⁻¹ (VW), 1769±4 cm⁻¹ (VW), 2786±4 cm⁻¹ (W), 2834±4 cm⁻¹ (W), 2875±4 cm⁻¹ (W), 2899±4 cm⁻¹ (VW), 2928±4 cm⁻¹ (W), 2943±4 cm⁻¹ (W), 2953±4 cm⁻¹ (VW), 2970±4 cm⁻¹ (VW), 2993±4 cm⁻¹ (VW), 3011±4 cm⁻¹ (VW), 3062±4 cm⁻¹ (W) and 3087±4 cm⁻¹ (VW).

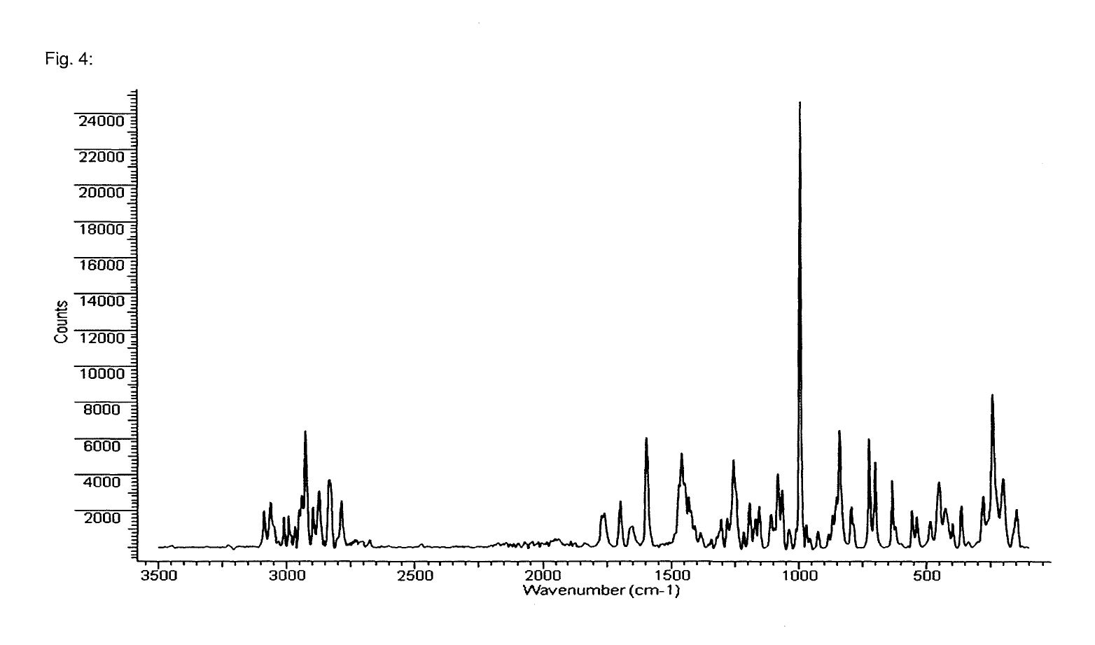

The peak intensities are described by VW (very weak), W (weak), M (medium) and VS (very strong). The actual Raman spectrum of crystalline modification A is shown in FIG. 4.

Another aspect of the present invention relates to a method for the production of the crystalline modification A described above comprising the step of
(a) dissolving (1RS,3RS,6RS)-6-dimethylaminomethyl-1-(3-methoxyphenyl)-cyclohexan-1,3-diol in an ester.
   Preferably, in the method according to the invention the ester is ethyl acetate.

Preferably, in the method according to the invention, step (a) is carried out at a temperature not higher than 80° C., more preferably not higher than 60° C., even more preferably not higher than 40° C. and/or in particular in a temperature range of 20-40° C.

Preferably the method according to the invention comprises the step
(b) precipitation of (1RS,3RS,6RS)-6-dimethylaminomethyl-1-(3-methoxyphenyl)-cyclohexan-1,3-diol or one of its individual enantiomers from the solution obtained in step (a).
   Suitable methods of precipitation are known to persons skilled in the art. Preferably, in the method according to the invention, step (b) may be carried out by reducing the volume of the solution obtained according to step (a) and/or by cooling of the solution, preferably to a temperature of at most 15° C., more preferably at most 10° C., even more preferably at most 4-8° C. and/or by cooling of the solution, preferably to a temperature of at least 10° C., more preferably at least 30° C., even more preferably at least 60° C. below the temperature according to step (a).

Preferably, in the method according to the invention, after the precipitation in step (b), all other steps are carried out at a temperature between 40 and 0° C., preferably between 35 and 5° C., more preferably between 25 and 15° C.

Preferably the method according to the invention may comprise the step (c) drying of the precipitate obtained in step (b).

Preferably, in the method according to the invention, step (c) takes place under air. However, drying under vacuum, more preferably at a vacuum of 1.0 to 900 mbar, even more preferably at a vacuum of 10 to 500 mbar, and in particular at a vacuum of 20 to 200 mbar is also possible.

Preferably, in the method according to the invention, step (c) takes place in a temperature range from 0 to 60° C., preferably from 10° C. to 50° C. more preferably from 20 to 40° C.

Another aspect of the present invention relates to a method for the production of the crystalline modification A described above comprising the step of
(a-1) dissolving (1RS,3RS,6RS)-6-dimethylaminomethyl-1-(3-methoxyphenyl)-cyclohexan-1,3-diol in an ester.

Preferably, in the method according to the invention the ester is ethyl acetate.

Preferably, in the method according to the invention, step (a-1) is carried out at a temperature not higher than 80° C., more preferably not higher than 60° C., even more preferably not higher than 40° C. and/or in particular in a temperature range of 20-40° C.

Preferably the method according to the invention comprises the step (b-1) evaporating off the solvent of the solution obtained in step (a-1). Suitable methods for evaporating the solvent are known to persons skilled in the art. Preferably, in the method according to the invention, the solvent is evaporated off in air or air flow.

Another aspect of the present invention relates to a method for the production of the crystalline modification A described above comprising the step of
(a-2) dissolving (1RS,3RS,6RS)-6-dimethylaminomethyl-1-(3-methoxyphenyl)-cyclohexan-1,3-diol in an alcohol.

Preferably, in the method according to the invention the alcohol is selected from the group consisting of methanol, ethanol, 1-propanol and 2-propanol, whereby methanol is particularly preferred.

Preferably, in the method according to the invention, step (a-2) is carried out at a temperature not higher than 80° C., more preferably not higher than 60° C., even more preferably not higher than 40° C. and/or in particular in a temperature range of 20-40° C.

Preferably the method according to the invention comprises the step (b-2) evaporating off the solvent of the solution obtained in step (a-2). Suitable methods for evaporating the solvent are known to persons skilled in the art. Preferably, in the method according to the invention, the solvent is evaporated off in air or air flow.

Another aspect of the present invention relates to a method for the production of the crystalline modification A described above comprising the step of
(a-3) dissolving (1RS,3RS,6RS)-6-dimethylaminomethyl-1-(3-methoxyphenyl)-cyclohexan-1,3-diol in an alcohol.

Preferably, in the method according to the invention the alcohol is selected from the group consisting of methanol, ethanol, 1-propanol and 2-propanol, whereby methanol is particularly preferred.

Preferably, in the method according to the invention, step (a-3) is carried out at a temperature not higher than 80° C., more preferably not higher than 60° C., even more preferably not higher than 40° C. and/or in particular in a temperature range of 20-40° C.

Preferably the method according to the invention comprises the step
(b-3) precipitation of (1RS,3RS,6RS)-6-dimethylaminomethyl-1-(3-methoxyphenyl)-cyclohexan-1,3-diol or one of its individual enantiomers from the solution obtained in step (a-3).
   Suitable methods of precipitation are known to persons skilled in the art. Preferably, in the method according to the invention, step (b-3) may be carried out by reducing the volume of the solution obtained according to step (a) and/or by cooling of the solution, preferably to a temperature of at most 15° C., more preferably at most 10° C., even more preferably at most 4-8° C. and/or by cooling of the solution, preferably to a temperature of at least 10° C., more preferably at least 30° C., even more preferably at least 60° C. below the temperature according to step (a-3).

Preferably, in the method according to the invention, after the precipitation in step (b-3), all other steps are carried out at a temperature between 40 and 0° C., preferably between 35 and 5° C., more preferably between 25 and 15° C.

Preferably the method according to the invention may comprise the step (c-3) drying of the precipitate obtained in step (b-3).

Preferably, in the method according to the invention, step (c-3) takes place under air. However, drying under vacuum, more preferably at a vacuum of 1.0 to 900 mbar, even more preferably at a vacuum of 10 to 500 mbar, and in particular at a vacuum of 20 to 200 mbar is also possible.

Preferably, in the method according to the invention, step (c-3) takes place in a temperature range from 0 to 60° C., preferably from 10° C. to 50° C. more preferably from 20 to 40° C.

A further aspect of the present invention relates to a crystalline modification A that can be obtained as described above.

Crystalline modification A is the most thermodynamically stable form, in particular in the temperature range of −20° C. to 120° C., preferably 0-100° C., more preferably 25-75° C. Accordingly, it may generally be obtained by slower crystallisation and/or by slower evaporation techniques. The thermodynamic stability is important. By using the most stable modification in a medicament it may specifically be ensured that, during storage, no polymorphic conversion of the active ingredient in the pharmaceutical formulation takes place. This is advantageous, because otherwise the properties of the medicament could change as a consequence of a conversion of a less stable modification into a more stable modification. In relation to the pharmacological properties of an administration form, this could lead for example to the solubility of the active ingredient changing, accompanied by a change in the release characteristics and thus also a change in the bioavailability. Lastly, this could result in inadequate storage stability of the medicament.

A further subject of the present invention relates to a crystalline modification B (polymorph B). Said crystalline modification B according to the present invention has at least one X-ray diffraction peak selected from the group consisting of 11.35±0.20 (2Θ) and 24.30±0.20 (2Θ).

Preferably, the crystalline modification B according to the present invention may additionally have at least one X-ray diffraction peak selected from the group consisting of 12.75±0.20 (2Θ), 14.04±0.20 (2Θ), 16.51±0.20 (2Θ), 18.79±0.20 (2Θ), 19.74±0.20 (2Θ), 20.09±0.20 (2Θ) and 21.20±0.20 (2Θ).

The crystalline modification B according to the present invention may further be characterized in that as well as the one or more X-ray diffraction peaks selected from the group consisting of 11.35±0.20 (2Θ) and 24.30±0.20 (2Θ), and optionally one or more X-ray diffraction peaks selected from the group consisting of 12.75±0.20 (2Θ), 14.04±0.20 (2Θ), 16.51±0.20 (2Θ), 18.79±0.20 (2Θ), 19.74±0.20 (2Θ), 20.09±0.20 (2Θ) and 21.20±0.20 (2Θ), it additionally has at least one X-ray diffraction peak selected from the group consisting of 15.23±0.20 (2Θ), 19.20±0.20 (2Θ), 21.42±0.20 (2Θ), 23.69±0.20 (2Θ), 23.76±0.20 (2Θ), 24.30±0.20 (2Θ), 25.66±0.20 (2Θ), 25.74±0.20 (2Θ), 25.84±0.20 (2Θ), 28.30±0.20 (2Θ) and 31.81±0.20 (2Θ).

Furthermore, the crystalline modification B according to the invention may be characterized in that as well as one or more X-ray diffraction peaks selected from the group consisting of 11.35±0.20 (2Θ) and 24.30±0.20 (2Θ) and optionally one or more X-ray diffraction peaks selected from the group consisting of 12.75±0.20 (2Θ), 14.04±0.20 (2Θ), 16.51±0.20 (2Θ), 18.79±0.20 (2Θ), 19.74±0.20 (2Θ), 20.09±0.20 (2Θ) and 21.20±0.20 (2Θ) and optionally one or more X-ray diffraction peaks selected from the group consisting of 15.23±0.20 (2Θ), 19.20±0.20 (2Θ), 21.42±0.20 (2Θ), 23.69±0.20 (2Θ), 23.76±0.20 (2Θ), 24.30±0.20 (2Θ), 25.66±0.20 (2Θ), 25.74±0.20 (2Θ), 25.84±0.20 (2Θ), 28.30±0.20 (2Θ), 31.81±0.20 (2Θ) it additionally has at least one X-ray diffraction peak selected from the group consisting of 9.72±0.20 (2Θ), 12.79±0.20 (2Θ), 22.82±0.20 (2Θ), 26.55±0.20 (2Θ), 26.77±0.20 (2Θ), 27.07±0.20 (2Θ), 27.83±0.20 (2Θ), 28.07±0.20 (2Θ), 28.49±0.20 (2Θ), 29.44±0.20 (2Θ), 29.74±0.20 (2Θ), 30.21±0.20 (2Θ), 30.27±0.20 (2Θ), 30.62±0.20 (2Θ), 30.74±0.20 (2Θ), 31.96±0.20 (2Θ), 32.01±0.20 (2Θ), 32.09±0.20 (2Θ), 33.18±0.20 (2Θ), 33.39±0.20 (2Θ), 33.94±0.20 (2Θ), 34.01±0.20 (2Θ), 34.25±0.20 (2Θ), 34.52±0.20 (2Θ), 34.85±0.20 (2Θ), 35.37±0.20 (2Θ), 35.55±0.20 (2Θ), 35.70±0.20 (2Θ), 35.83±0.20 (2Θ), 37.21±0.20 (2Θ), 37.29±0.20 (2Θ), 37.63±0.20 (2Θ), 38.12±0.20 (2Θ), 38.20±0.20 (2Θ), 38.48±0.20 (2Θ), 38.50±0.20 (2Θ), 38.96±0.20 (2Θ), 39.04±0.20 (2Θ), 39.47±0.20 (2Θ) and 39.97±0.20 (2Θ).

The crystalline modification B according to the invention may also be characterized in that as well as the at least one X-ray diffraction peak selected from the group consisting of 11.35±0.20 (2Θ) and 24.30±0.20 (2Θ) and optionally one or more X-ray diffraction peak selected from the group consisting of 12.75±0.20 (2Θ), 14.04±0.20 (2Θ), 16.51±0.20 (2Θ), 18.79±0.20 (2Θ), 19.74±0.20 (2Θ), 20.09±0.20 (2Θ) and optionally one or more X-ray diffraction peak selected from the group consisting of 15.23±0.20 (2Θ), 19.20±0.20 (2Θ), 21.42±0.20 (2Θ), 23.69±0.20 (2Θ), 23.76±0.20 (2Θ), 24.30±0.20 (2Θ), 25.66±0.20 (2Θ), 25.74±0.20 (2Θ), 25.84±0.20 (2Θ), 28.30±0.20 (2Θ), 31.81±0.20 (2Θ), and optionally one or more X-ray diffraction peak selected from the group consisting of 9.72±0.20 (2Θ), 12.79±0.20 (2Θ), 22.82±0.20 (2Θ), 26.55±0.20 (2Θ), 26.77±0.20 (2Θ), 27.07±0.20 (2Θ), 27.83±0.20 (2Θ), 28.07±0.20 (2Θ), 28.49±0.20 (2Θ), 29.44±0.20 (2Θ), 29.74±0.20 (2Θ), 30.21±0.20 (2Θ), 30.27±0.20 (2Θ), 30.62±0.20 (2Θ), 30.74±0.20 (2Θ), 31.96±0.20 (2Θ), 32.01±0.20 (2Θ), 32.09±0.20 (2Θ), 33.18±0.20 (2Θ), 33.39±0.20 (2Θ), 33.94±0.20 (2Θ), 34.01±0.20 (2Θ), 34.25±0.20 (2Θ), 34.52±0.20 (2Θ), 34.85±0.20 (2Θ), 35.37±0.20 (2Θ), 35.55±0.20 (2Θ), 35.70±0.20 (2Θ), 35.83±0.20 (2Θ), 37.21±0.20 (2Θ), 37.29±0.20 (2Θ), 37.63±0.20 (2Θ), 38.12±0.20 (2Θ), 38.20±0.20 (2Θ), 38.48±0.20 (2Θ), 38.50±0.20 (2Θ), 38.96±0.20 (2Θ), 39.04±0.20 (2Θ), 39.47±0.20 (2Θ) and 39.97±0.20 (2Θ) it additionally has at least one X-ray diffraction peak selected from the group consisting of 19.50±0.20 (2Θ), 20.94±0.20 (2Θ), 22.24±0.20 (2Θ), 25.19±0.20 (2Θ), 27.23±0.20 (2Θ), 27.42±0.20 (2Θ), 28.52±0.20 (2Θ), 33.14±0.20 (2Θ), 33.62±0.20 (2Θ), 34.11±0.20 (2Θ), 34.51±0.20 (2Θ), 35.15±0.20 (2Θ), 35.54±0.20 (2Θ), 35.89±0.20 (2Θ), 36.11±0.20 (2Θ), 36.30±0.20 (2Θ), 38.06±0.20 (2Θ), 38.75±0.20 (2Θ), 38.91±0.20 (2Θ), 39.01±0.20 (2Θ), 39.08±0.20 (2Θ) and 39.60±0.20 (2Θ).

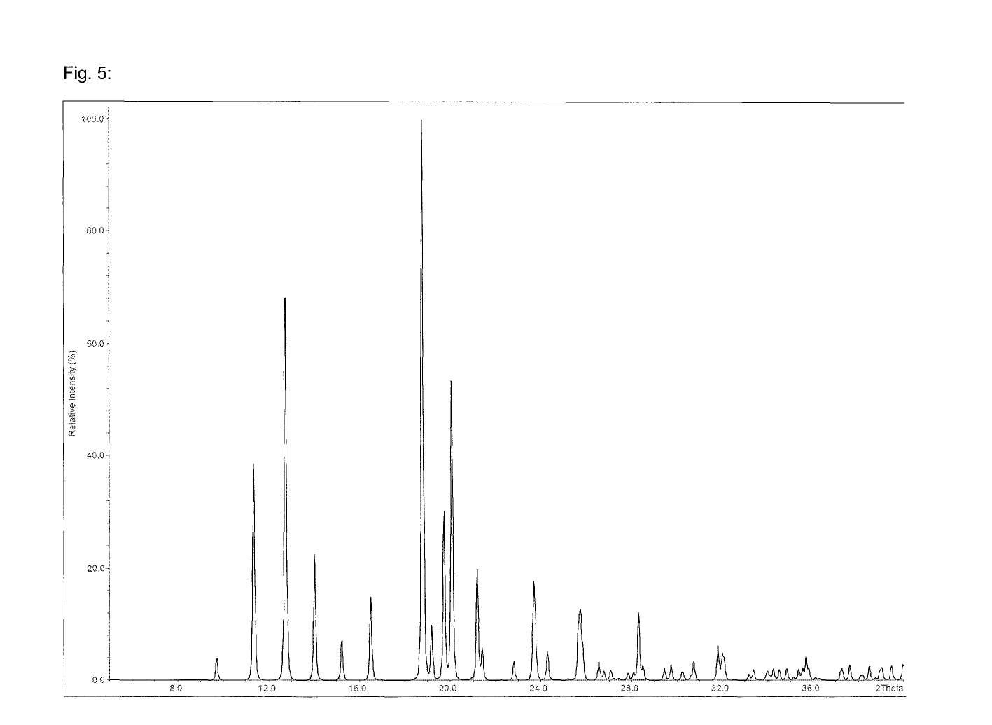

A calculated diffractogram of crystalline modification B is shown in FIG. 5.

In DSC analyses, the crystalline modification B according to the present invention preferably exhibits an endothermal event with a peak temperature at 109-120° C., more preferably at 110-119° C., even more preferably at 111-118° C. and in particular at 112-115° C.

Another aspect of the present invention relates to a method for the production of the crystalline modification B described above comprising the step of
(a) dissolving (1RS,3RS,6RS)-6-dimethylaminomethyl-1-(3-methoxyphenyl)-cyclohexan-1,3-diol in a chlorinated hydrocarbon.

Preferably, in the method according to the invention the chlorinated hydrocarbon is dichloromethane.

Preferably, in the method according to the invention, step (a) is carried out at a temperature not higher than 80° C., more preferably not higher than 60° C., even more preferably not higher than 40° C. and/or in particular in a temperature range of 20-40° C. Preferably, in the method according to the invention, step (a) is carried out under application of energy, e.g. via ultrasound.

Preferably the method according to the invention comprises the step (b) adding an antisolvent to the solution obtained in step (a).

An antisolvent as used herein designates a organic medium in which (1RS,3RS,6RS)-6-dimethylaminomethyl-1-(3-methoxyphenyl)-cyclohexan-1,3-diol or its individual enantiomer shows a lower solubility than in the chlorinated hydrocarbon used in step (a), for example, n-hexane or n-pentane. The amount of the antisolvent can preferably be selected in such a manner that upon its addition precipation of the dissolved component begins.

The temperature of the solution at which the antisolvent is added and the temperature of the antisolvent that is added can preferably be selected in such a manner that upon its addition precipation of the dissolved component begins immediately. Preferably there may be a difference between the temperature of the solution and the temperature of the antisolvent of at least 10° C., more preferably of at least 15° C., yet more preferably of at least 20° C., whereby it may be preferred that the solution has the higher temperature and the antisolvent the lower temperature.

Preferably the method according to the invention comprises the step:
(c) precipitation of (1RS,3RS,6RS)-6-dimethylaminomethyl-1-(3-methoxyphenyl)-cyclohexan-1,3-diol or one of its individual enantiomers from the solution obtained in step (a) or in step (b).

Suitable methods of initiating immediate precipitation are known to persons skilled in the art. Preferably, in the method according to the invention, step (c) is carried out by cooling the solution. Preferably it is cooled rapidly to a temperature of at most 15° C., more preferably at most 10° C., even more preferably at most 4-8° C. Rapid cooling can be realised e.g. by transferring the vial or flask with the solution into an icebath or into a suspension of dry ice in methanol.

Preferably, in the method according to the invention, after the precipitation in step (c), all other steps are carried out at a temperature between 40 and 0° C., preferably between 35 and 5° C., more preferably between 25 and 15° C.

Preferably the method according to the invention further comprises the step (d) drying of the precipitate obtained in step (c).

Preferably, in the method according to the invention, step (d) takes place under air. However, drying under vacuum, more preferably at a vacuum of 1.0 to 900 mbar, even more preferably at a vacuum of 10 to 500 mbar, and in particular at a vacuum of 20 to 200 mbar is also possible.

Preferably, in the method according to the invention, step (d) takes place in a temperature range from 0 to 60° C., preferably from 10° C. to 50° C. more preferably from 20 to 40° C.

A further aspect of the present invention relates to a crystalline modification B that can be obtained as described above.

Another aspect of the present invention relates to a method for the production of the crystalline modification B described above comprising the step of
(a-1) dissolving (1RS,3RS,6RS)-6-dimethylaminomethyl-1-(3-methoxyphenyl)-cyclohexan-1,3-diol in an ether.

Preferably, in the method according to the invention the ether is selected from the group consisting of diethylether, diisopropylether and tert-Butylmethylether.

Preferably, in the method according to the invention, step (a) is carried out at a temperature not higher than 80° C., more preferably not higher than 60° C., even more preferably not higher than 40° C. and/or in particular in a temperature range of 20-40° C.

Preferably the method according to the invention comprises the step
(b-1) evaporating off the solvent of the solution obtained in step (a-1).

Suitable methods of evaporating the solvent are known to persons skilled in the art. Preferably, in the method according to the invention, the solvent is evaporated off in air or air flow, more preferably by evaporating the solvent applying a vacuum.

It seems that generally crystalline modification B can be obtained by faster crystallisation and/or at higher temperatures.

A further subject of the present invention relates to a crystalline modification C (polymorph C). Said crystalline modification C according to the present invention has at least one X-ray diffraction peak selected from the group consisting of 9.05±0.20 (2Θ), 14.64±0.20 (2Θ), 15.83±0.20 (2Θ) and 16.07±0.20 (2Θ).

Preferably, the crystalline modification C according to the present invention may additionally have at least one X-ray diffraction peak selected from the group consisting of 15.47±0.20 (2Θ), 16.84±0.20 (2Θ), 18.07±0.20 (2Θ), 19.64±0.20 (2Θ), 20.23±0.20 (2Θ), 21.04±0.20 (2Θ), 21.49±0.20 (2Θ), 22.04±0.20 (2Θ), 24.79±0.20 (2Θ), 25.69±0.20 (2Θ), 27.80±0.20 (2Θ), 28.22±0.20 (2Θ) and 31.17±0.20 (2Θ).

Furthermore, the crystalline modification C according to the present invention may be characterized in that as well as one or more X-ray diffraction peaks selected from the group consisting of 9.05±0.20 (2Θ), 14.64±0.20 (2Θ), 15.83±0.20 (2Θ) and 16.07±0.20 (2Θ) and optionally one or more X-ray diffraction peaks selected from the group consisting of 15.47±0.20 (2Θ), 16.84±0.20 (2Θ), 18.07±0.20 (2Θ), 19.64±0.20 (2Θ), 20.23±0.20 (2Θ), 21.04±0.20 (2Θ), 21.49±0.20 (2Θ), 22.04±0.20 (2Θ), 24.79±0.20 (2Θ), 25.69±0.20 (2Θ), 27.80±0.20 (2Θ), 28.22±0.20 (2Θ) and 31.17±0.20 (2Θ) it additionally has at least one X-ray diffraction peak selected from the group consisting of 12.54±0.20 (2Θ), 15.02±0.20 (2Θ), 17.77±0.20 (2Θ), 24.94±0.20 (2Θ), 25.18±0.20 (2Θ), 25.82±0.20 (2Θ), 26.34±0.20 (2Θ), 26.82±0.20 (2Θ), 29.25±0.20 (2Θ), 29.46±0.20 (2Θ), 29.89±0.20 (2Θ), 30.07±0.20 (2Θ), 34.00±0.20 (2Θ), 35.90±0.20 (2Θ), 36.34±0.20 (2Θ) and 39.12±0.20 (2Θ).

Furthermore, the crystalline modification C according to the present invention may be characterized in that as well as one or more X-ray diffraction peaks selected from the group consisting of 9.05±0.20 (2Θ), 14.64±0.20 (2Θ), 15.83±0.20 (2Θ) and 16.07±0.20 (2Θ) and optionally one or more X-ray diffraction peaks selected from the group consisting of 15.47±0.20 (2Θ), 16.84±0.20 (2Θ), 18.07±0.20 (2Θ), 19.64±0.20 (2Θ), 20.23±0.20 (2Θ), 21.04±0.20 (2Θ), 21.49±0.20 (2Θ), 22.04±0.20 (2Θ), 24.79±0.20 (2Θ), 25.69±0.20 (2Θ), 27.80±0.20 (2Θ), 28.22±0.20 (2Θ) and 31.17±0.20 (2Θ) and optionally one or more X-ray diffraction peaks selected from the group of 12.54±0.20 (2Θ), 15.02±0.20 (2Θ), 17.77±0.20 (2Θ), 24.94±0.20 (2Θ), 25.18±0.20 (2Θ), 25.82±0.20 (2Θ), 26.34±0.20 (2Θ), 26.82±0.20 (2Θ), 29.25±0.20 (2Θ), 29.46±0.20 (2Θ), 29.89±0.20 (2Θ), 30.07±0.20 (2Θ), 34.00±0.20 (2Θ), 35.90±0.20 (2Θ), 36.34±0.20 (2Θ) and 39.12±0.20 (2Θ) it additionally comprises at least one X-ray diffraction peak selected from the group consisting of 10.04±0.20 (2Θ), 23.78±0.20 (2Θ), 30.31±0.20 (2Θ), 30.64±0.20 (2Θ), 32.47±0.20 (2Θ), 32.94±0.20 (2Θ), 33.21±0.20 (2Θ), 34.40±0.20 (2Θ), 38.13±0.20 (2Θ) and 39,31±0.20 (2Θ).

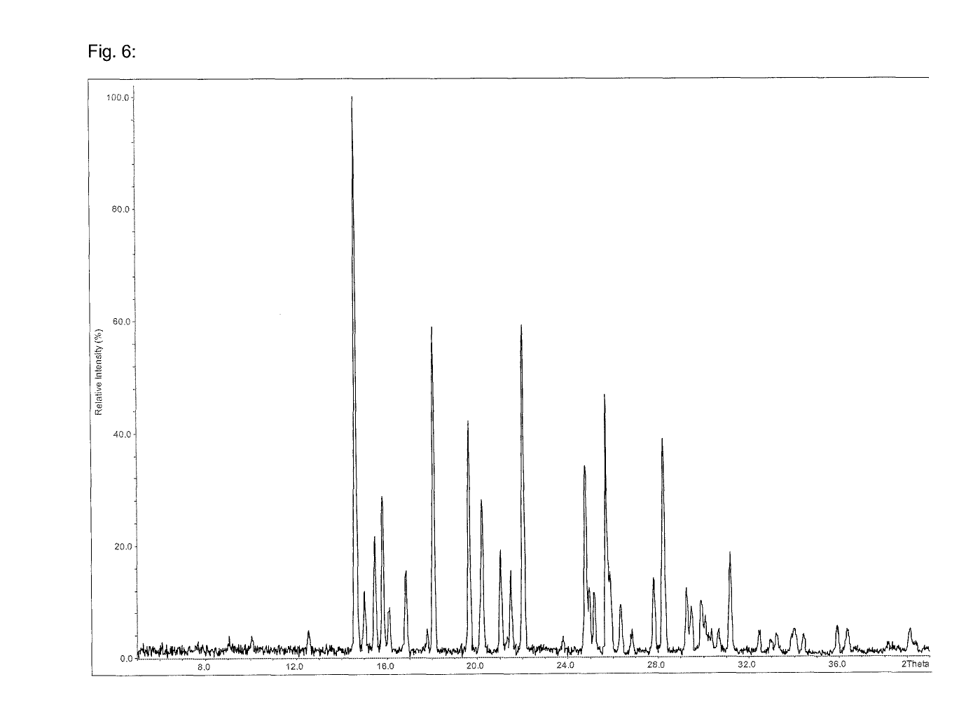

A measured diffractogram of crystalline modification C is shown in FIG. 6. In DSC analyses, the crystalline modification C according to the present invention preferably exhibits an endothermal event with a peak temperature at 113-124° C., more preferably at 114-123° C., even more preferably at 115-122° C. and in particular at 116-121° C.

Another aspect of the present invention relates to a method for the production of the crystalline modification C described above comprising the step of
(a) dissolving (1RS,3RS,6RS)-6-dimethylaminomethyl-1-(3-methoxyphenyl)-cyclohexan-1,3-diol in a ketone.
   Preferably, in the method according to the invention the ketone is selected from the group consisting of acetone, butan-2-one, pentan-2-one, pentan-3-one, hexan-2-one and hexan-3-one. Acetone is particularly preferred.

Preferably, in the method according to the invention, step (a) is carried out at a temperature not higher than 80° C., more preferably not higher than 60° C., even more preferably not higher than 40° C. and/or in particular in a temperature range of 20-40° C.

Preferably, in the method according to the invention, step (a) is carried out under application of energy, e.g. via ultrasound.

Preferably the method according to the invention comprises the step
(b) adding an antisolvent to the solution obtained in step (a).
   An antisolvent as used herein designates a organic medium in which (1RS,3RS,6RS)-6-dimethylaminomethyl-1-(3-methoxyphenyl)-cyclohexan-1,3-diol or one of its enantiomers shows a lower solubility than in the ketone used in step (a), for example, n-hexane or n-pentane. The amount of the antisolvent can preferably be selected in such a manner that upon its addition precipation of the dissolved component begins.

Preferably the method according to the invention comprises the step
(c) precipitation of (1RS,3RS,6RS)-6-dimethylaminomethyl-1-(3-methoxyphenyl)-cyclohexan-1,3-diol or one of its individual enantiomers from the solution obtained in step (a) or in step (b).
   Suitable methods of precipitation are known to persons skilled in the art. Preferably, in the method according to the invention, step (c) is carried out by cooling the solution, preferably to a temperature of at most 15° C., more preferably at most 10° C., even more preferably at most 4-8° C. and/or by cooling of the solution, preferably to a temperature of at least 10° C., more preferably at least 30° C., even more preferably at least 60° C. below the temperature according to step (a) or step (b).

Preferably, in the method according to the invention, after the precipitation in step (c), all other steps are carried out at a temperature between 40 and 0° C., preferably between 35 and 5° C., more preferably between 25 and 15° C.

Preferably the method according to the invention comprises the step
(d) drying of the precipitate obtained in step (c).

Preferably, in the method according to the invention, step (d) takes place under air. However, drying under vacuum, more preferably at a vacuum of 1.0 to 900 mbar, even more preferably at a vacuum of 10 to 500 mbar, and in particular at a vacuum of 20 to 200 mbar is also possible.

Preferably, in the method according to the invention, step (d) takes place in a temperature range from 0 to 60° C., preferably from 10° C. to 50° C. more preferably from 20 to 40° C.

A further aspect of the present invention relates to a crystalline modification C that can be obtained as described above.

The modifications A, B and C according to the invention may optionally also form co-crystals and solvates. These are all included within the scope of the present invention.

It is known to persons skilled in the art, e.g. from Jane Li et al. in J. Pharm. Sci., 1999, Vol. 88(3),pages 337-346 that enantiomers form identical crystalline modifications (polymorphs) and accordingly show identical X-ray-diffractograms and Raman spectra. Thus, the present invention comprises crystalline modifications of both enantiomers (1R,3R,6R)-6-dimethylaminomethyl-1-(3-methoxyphenyl)-cyclohexan-1,3-diol and (1S,3S,6S)-6-dimethylaminomethyl-1-(3-methoxyphenyl)-cyclohexan-1,3-diol as mixtures thereof, such as the racemate (1RS,3RS,6RS)-6-dimethylaminomethyl-1-(3-methoxyphenyl)-cyclohexan-1,3-diol.

It was surprisingly found that the compound (1RS,3RS,6RS)-6-dimethylaminomethyl-1-(3-methoxyphenyl)-cyclohexan-1,3-diol forms a conglomerate, i.e. the resulting individual crystals do not comprise a racemic composition but the individual enantiomers.

Accordingly, the individual enantiomers (1R,3R,6R)-6-dimethylaminomethyl-1-(3-methoxyphenyl)-cyclohexan-1,3-diol and (1S,3S,6S)-6-dimethylaminomethyl-1-(3-methoxyphenyl)-cyclohexan-1,3-diol may be obtained via preferential crystallisation from a solution comprising these compounds, which is an effective and cost-effective method and allows for the production of the pure enantiomers at different scales.

Thus, in yet a further aspect the present invention relates to a process for obtaining or enriching (1R,3R,6R)-6-dimethylaminomethyl-1-(3-methoxyphenyl)-cyclohexane-1,3-diol or (1S,3S,6S)-6-dimethylaminomethyl-1-(3-methoxyphenyl)-cyclohexane-1,3-diol, which comprises the step of cooling a solution comprising these compounds.

The process of the invention process may be carried out starting from a solution that contains the individual enantiomers in a racemic mixture or a non-racemic mixture, whereby the latter is preferred. A non-racemic mixture can contain the enantiomers in any possible ratio, for example, in a ratio of 60±5:40±5; 70±5:30±5; 80±5:20±5 or 90±5:10±5. Preferably, the non-racemic mixture may contain at least 55%, preferably at least 60% of one enantiomer, even more preferably at least 70% of one enantiomer, preferably the enantiomer that is to be obtained or enriched.

Suitable media for carrying out the process of the invention are any conventional organic solvents known to the person skilled in the art or mixtures of two or more of such solvents, for example, alcohols such as methanol, ethanol, 1-propanol and 2-propanol, esters such as ethyl acetate, ketones such as acetone and ethylmethyl ketone, ethers such as diethyl ether, diisopropylether, 1,4-dioxane and tetrahydrofuran, nitriles such as acetonitrile, chlorinated hydrocarbons such as dichloromethane, aromatic hydrocarbons such as toluene, and also dimethyl formamide and dimethyl sulfoxide. Preferably ethers such as diisopropylether may be used in the process according to the present invention.

The solution may be seeded with crystalline material of one of the enantiomers, preferably of the enantiomer that is to be obtained or enriched. Such crystals of the enantiomer may be obtained by methods well-known to those skilled in the art, for example, via chromatographic methods such as HPLC or via diastereomeric salt formation and subsequent release and crystallisation of the free base.

The solution may be cooled by a temperature difference that varies over a broad range, e.g. at least 75° C., preferably at least 65° C., more preferably at least 55° C., even more preferably at least 50° C. and in particular 40-50° C. For example, the solution may be cooled from a temperature of 50-65° C. to a room temperature, for example, 15-25° C.

In another aspect the present invention relates to the use of a crystalline modification as described herein for the treatment of pain. The term pain as used herein preferably includes but is not limited to pain selected from the group consisting of inflammatory pain, postoperative pain, neuropathic pain, diabetic neuropathic pain, acute pain, chronic pain, visceral pain, migraine pain and cancer pain. Different types of pain associated with arthrosis are described, for example, in WO 2008/138558 the respective contents of which hereby being incorporated by reference and forming part of the disclosure of the present invention.

In another aspect the present invention relates to a pharmaceutical composition comprising a crystalline modification as described herein and optionally one or more suitable additives and/or adjuvants such as described below. Preferably the pharmaceutical composition may be used for the treatment of pain.

In still another aspect the present invention relates to a medicament comprising a crystalline modification as described herein. In a preferred embodiment, the medicament is a solid drug form. The medicament is preferably manufactured for oral administration. However, other forms of administration are also possible, e.g. for buccal, sublingual, transmucosal, rectal, intralumbal, intraperitoneal, transdermal, intravenous, intramuscular, intragluteal, intracutaneous and subcutaneous administration.

Depending on the configuration, the medicament (dosage form) preferably contains suitable additives and/or adjuvants. Suitable additives and/or adjuvants in the sense of the invention include all substances known to persons skilled in the art for the preparation of galenic formulations. The choice of these adjuvants and also the quantities to be used depend on how the medication is to be administered, i.e. orally, intravenously, intraperitoneally, intradermally, intramuscularly, intranasally, buccally or locally.

Preparations suitable for oral administration are those in the form of tablets, chewable tablets, lozenges, capsules, granules, drops, liquids or syrups, and those suitable for parenteral, topical and inhalatory administration are solutions, suspensions, easily reconstituted dry preparations and sprays. A further possibility is suppositories for rectal administration. Examples of suitable percutaneous forms of administration include application in a depot in dissolved form, a patch or a plaster, optionally with the addition of agents which promote skin penetration.

Examples of adjuvants and additives for oral forms of administration include disintegrants, lubricants, binders, fillers, mold-release agents, solvents, flavorings, sugar, in particular carriers, diluents, coloring agents, antioxidants etc.

Waxes or fatty acid esters, amongst others, can be used for suppositories and carrier substances, preservatives, suspension aids etc. can be used for parenteral forms of application.

Adjuvants may include, for example: water, ethanol, 2-propanol, glycerine, ethylene glycol, propylene glycol, polyethylene glycol, polypropylene glycol, glucose, fructose, lactose, saccharose, dextrose, molasses, starch, modified starch, gelatine, sorbitol, inositol, mannitol, microcrystalline cellulose, methyl cellulose, carboxymethyl-cellulose, cellulose acetate, shellac, cetyl alcohol, polyvinylpyrrolidone, paraffins, waxes, natural and synthetic rubbers, acacia gum, alginates, dextran, saturated and unsaturated fatty acids, stearic acid, magnesium stearate, zinc stearate, glyceryl stearate, sodium lauryl sulfate, edible oils, sesame oil, coconut oil, peanut oil, soybean oil, lecithin, sodium lactate, polyoxyethylene and propylene fatty acid esters, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talc, kaolin, pectin, crospovidon, agar and bentonite.

The production of these medicaments and pharmaceutical compositions is carried out using means, devices, methods and processes that are well known in the art of pharmaceutical technology, as described, for example, in “Remington's Pharmaceutical Sciences”, A. R. Gennaro, 17th ed., Mack Publishing Company, Easton, Pa. (1985), in particular in part 8, chapters 76 to 93.

Thus, for example, for a solid formulation such as a tablet, the active substance of the drug can be granulated with a pharmaceutical carrier substance, e.g. conventional tablet constituents such as cornstarch, lactose, saccharose, sorbitol, talc, magnesium stearate, dicalcium phosphate or pharmaceutically acceptable rubbers, and pharmaceutical diluents such as water, for example, in order to form a solid composition that contains the active substance in a homogenous dispersion. Homogenous dispersion is understood here to mean that the active substances are uniformly dispersed throughout the composition, so that this can be readily divided into identically effective standard dosage forms such as tablets, capsules, lozenges. The solid composition is then divided into standard dosage forms. The tablets or pills can also be coated or otherwise compounded to prepare a slow release dosage form. Suitable coating agents include polymeric acids and mixtures of polymeric acids with materials such as shellac, cetyl alcohol and/or cellulose acetate, for example.

In one embodiment of the present invention the crystalline modification as described herein is present in immediate release form.

In another embodiment of the present invention the crystalline modification as described herein is at least partially present in controlled-release form. In particular, the active ingredient can be released slowly from preparations that can be applied orally, rectally or percutaneously.

The medicament can preferably be manufactured for administration once daily, twice daily (bid), or three times daily, the once daily or twice daily administration (bid) being preferred.

As used herein the term “controlled release” refers to any type of release other than immediate release such as delayed release, sustained release, slow release, extended release and the like. These terms are well known to persons skilled in the art, as are the means, devices, methods and processes for obtaining each such type of release. A controlled release of the active ingredient can be achieved, for example, by retardation using a matrix, a coating or osmotically active release systems such as described for axomadol in WO 2005/009329, for example, the entire disclosure of which is hereby incorporated by reference and forms part of the present disclosure.

In another embodiment of the present invention
the medicament is manufactured for oral administration; and/or
the medicament is a solid and/or compressed and/or film-coated drug form; and/or
the medicament releases the microcrystalline modification as described herein slowly from a matrix; and/or
the medicament contains the microcrystalline modification in a quantity of 0.001 to 99.999% by wt., more preferred 0.1 to 99.9% by wt., still more preferred 1.0 to 99.0% by wt., even more preferred 2.5 to 80% by wt., most preferred 5.0 to 50% by wt. and in particular 7.5 to 40% by wt., based on the total weight of the medicament; and/or
the medicament contains a pharmaceutically compatible carrier and/or pharmaceutically compatible adjuvants; and/or
the medicament has a total mass in the range of 25 to 2000 mg, more preferably 50 to 1800 mg, still more preferably 60 to 1600 mg, more preferably 70 to 1400 mg, most preferred 80 to 1200 mg and in particular 100 to 1000 mg; and/or
the medicament is selected from the group comprising tablets, capsules, pellets and granules.

The medicament can be provided as a simple tablet and as a coated tablet (e.g. as film-coated tablet or lozenge). The tablets are usually round and biconvex, but oblong forms are also possible. Granules, spheres, pellets or microcapsules, which are contained in sachets or capsules or are compressed to form disintegrating tablets, are also possible.

In yet another one of its aspects, the present invention relates to the use of the crystalline modification as described herein for the production of a medicament. Preferably said medicament is suitable for the treatment of pain.

In still another one of its aspects, the present invention relates to the use of the crystalline modification as described herein for the treatment of pain. Furthermore, the present invention relates to a method for treating pain in a patient, preferably in a mammal, which comprises administering a pharmacologically effective amount of a crystalline modification as described herein to a patient.

The following examples serve to explain the invention in more detail, but should not be interpreted as limiting.

### EXAMPLES

### 1. Synthesis of Crystalline Modification A

1.1 27.69 g (87.66 mmol) of (1RS,3RS,6RS)-6-dimethylaminomethyl-1-(3-methoxyphenyl)-cyclohexan-1,3-diol hydrochloride were dissolved in 140 mL of destilled water and cooled to 25° C. At a temperature of 25° C. an aqueous solution of sodium hydroxide (32%) was added until a pH of 11 was obtained. Subsequent to the addition of 10 ml of said solution precipitation of a white, oily solid was observed, which was dissolved by adding 10 ml ethyl acetate. After the addition of 20 ml of the aqueous solution of sodium hydroxide (32%) a pH of 11 was obtained.

(1RS,3RS,6RS)-6-dimethylaminomethyl-1-(3-methoxyphenyl)-cyclohexan-1,3-diol was extracted with ethyl acetate, the resulting organic phase was dried over magnesium sulfate, filtered and evaporated in vacuo to about half its volume by means of a rotary evaporator. The resulting solution was kept at room temperature for 5 days, upon which large, colorless orthorhombic crystals were obtained, which were filtered of and washed with a small amount of cooled ethyl acetate (Yield 6.07 g). The remaining solution was evaporated to dryness to yield a light brown solid (Yield 16.49). The resulting products were characterized by ¹H-NMR spectroscopy. According to x-ray powder diffraction (XRPD) the product obtained was crystalline modification A.

1.2 10 mg of (1RS,3RS,6RS)-6-dimethylaminomethyl-1-(3-methoxyphenyl)-cyclohexan-1,3-diol obtained according to 1.1 were pestled, given into a vial and 1 ml of ethyl acetate was added. The vial was given into a Desyre reaction block that was vortexted by means of an 1KA-Vibrax® for 16 hours at room temperature and 400 rpm. Subsequently, the reaction block was kept in a refrigerator at 4° C. After 72 hours at 4° C. the vial still contained a clear solution and the reactor block was cooled to −18° C. After 72 hours at this temperature still no solid was observed. The sample was then kept at room temperature to evaporate off the solvent. After 3 days the sample was dried under nitrogen flow and crystals were obtained. (Yield 100%). According to x-ray powder diffraction (XRPD) the resulting product was crystalline modification A.

### 2. Synthesis of Crystalline Modification B

50.97 mg of (1RS,3RS,6RS)-6-dimethylaminomethyl-1-(3-methoxyphenyl)-cyclohexan-1,3-diol were given into a vial. At a temperature of 22° C. the solid was dissolved in 0.2 ml dichloromethane under ultrasound. Subsequently n-pentane was added until the beginning of precipitation could be observed (7 ml). The resulting clear solution was then kept in a refrigerator at a temperature of 4-8° C. After 28 hours crystalline material was obtained. After 52 hours the crystalline material was filtered off and dried under air flow. The product obtained was characterized by ¹H-NMR spectroscopy. According to x-ray powder diffraction (XRPD) the resulting product is crystalline modification B with minor impurities of crystalline modification A.

### 3. Synthesis of Crystalline Modification C

49.53 mg of (1RS,3RS,6RS)-6-dimethylaminomethyl-1-(3-methoxyphenyl)-cyclohexan-1,3-diol were given into a vial. At a temperature of 22° C. the solid was dissolved in 0.7 ml acetone under ultrasound. Subsequently n-hexane was added until the beginning of precipitation could be observed (7 ml). The resulting clear solution was then kept in a refrigerator at a temperature of 4-8° C. After 28 hours single crystals were obtained. After 52 hours the crystals were filtered off and dried under air flow. The product obtained was characterized by ¹H-NMR spectroscopy. According to x-ray powder diffraction (XRPD) the resulting product is crystalline modification C.

Moreover, two single crystals were used to determine the absolute configuration via X-ray single crystal diffraction. These two single crystals contained the different enantiomers (1R,3R,6R)-6-dimethylaminomethyl-1-(3-methoxyphenyl)-cyclohexan-1,3-diol and (1S,3S,6S)-6-dimethylaminomethyl-1-(3-methoxyphenyl)-cyclohexan-1,3-diol.

### 4. Crystallisation Experiments

In each of the following crystallisation experiments 40 mg of (1RS,3RS,6RS)-6-dimethylaminomethyl-1-(3-methoxyphenyl)-cyclohexan-1,3-diol were dissolved or suspended in the respective solvent as summerized in the in the following table 1:

TABLE 1

| | | Amount of | |
|---|---|---|---|
| Example | Solvent | Solvent/ml | Comment |
| 4-1 | methanol | 1 | |
| 4-2 | ethanol | 1 | |
| 4-3 | methanol/ethanol | 1 | |
| 4-4 | ethyl acetate | 2 | |
| 4-5 | diisopropyl ether | 6 | not dissolved |
| | | | completely |
| 4-6 | tetrahydrofuran | 2 | |

Subsequently, the samples were kept at room temperature. After five days some single crystals had formed. After an additional three days the crystals were separated and subjected to further analysis. Tetrahydrofuran did not yield clear crystals; diisopropyl ether yielded large crystals, methanol yielded good crystals, and ethyl acetate yielded small, clear crystals.

Surprisingly, the crystallisation experiments did not yield single crystals comprising the racemate (1RS,3RS,6RS)-6-dimethylaminomethyl-1-(3-methoxyphenyl)-cyclohexan-1,3-diol. Rather, the single crystals obtained in these cristallisation experiments consisted of the enantiomers as was verified via x-ray single crystal diffraction.

Thus, upon crystallisation, the compound (1RS,3RS,6RS)-6-dimethylamino-methyl-1-(3-methoxyphenyl)-cyclohexan-1,3-diol formed a conglomerate.

### Analysis—X-Ray Diffraction (XRD)

### (a) Measurements

### X-Ray Diffraction (XRD):

X-ray powder diffraction (XRPD) analyses were carried out in transmission geometry with a STOE Stadi P X-ray diffractometer, monochromatised CuKα₁ radiation being used by means of a germanium monocrystal. D-distances were calculated from the 2Θ values, establishing the wavelength of 1.540598 Å. In general, the 2Θ values have an error rate of ±0.2° in 2Θ. The experimental error in the d-distance values is therefore dependent on the location of the peak.

### b) Calculations

The peak tables and graphical representations of the diffractograms were produced on basis of the single crystal data using the programm WinXPow (THEO 1.11, version PKS_{—}2.01) of the company STOE. The parameters that were used for the calculations of the diffractograms as well as the peak lists in the computer program WinXPow are given in the following Table 2.

TABLE 2

| | Form A | Form B |
|---|---|---|
| | Single crystal | Single crystal |
| | obtained according | obtained according |
| Parameter | to example 4-1 | to example 4-5 |
| Formula | C₁₆H₂₅NO₃ | C₁₆H₂₅NO₃ |
| Laue Symmetry | Orthorhombic m m m | Monoclinic 2/m (b) |
| Lattice Type | Primitive | P 21 |
| Mol.Wt. | 279.42 | 279.42 |
| Z | 4.0 | 2.0 |
| Space Group | P 21 21 21 | P 21 |
| Radiation | Cu (1.540598) | Cu (1.540598) |
| Generate Full Pattern | Yes (box checked) | Yes (box checked) |
| 2Theta (Min, Max) | 0.1, 50.0 | 0.1, 50.0 |
| Cell Parameters A | 9.453 | 8.092 |
| Cell Parameters B | 10.118 | 10.727 |
| Cell Parameters C | 16.538 | 9.451 |
| Cell Parameters Alpha | 90.0 | 90.0 |
| Cell Parameters Beta | 90.0 | 105.751 |
| Cell Parameters Gamma | 90.0 | 90.0 |
| Geometry | Transmission | Transmission |
| Monochromator | Germanium | Germanium |
| Profile Function | Pearson | Pearson |
| Mu * T | 0.0 | 0.0 |
| Pearson Exponent | 2.0 | 2.0 |
| 2Theta (Min, Max, Step) | 0.1, 50.0, 0.02 | 0.1, 50.0, 0.02 |
| Halfwidth | 0.1, 0.0 | 0.1, 0.0 |
| Max. Intensity | 10000.0 | 10000.0 |
| Generate Alpha2 Peaks | No (box | No (box |
| | not checked) | not checked) |
| Constant Sample Area | No (box | No (box |
| | not checked) | not checked) |

### Peak Tables and Diffractograms

The values with regard to the relative intensities I (rel) were rounded. Thus, also peaks with an I(rel) of 0 may be seen in the diffractogram.

Due to the parameters chosen for the calculation of the diffractograms differences between the relative intensities (I(rel)) in the peak tables and the graphical representation of the calculated diffractogram may occur.

This is the case, for example, for form A: In the peak list the reflex at 16.71 (2 Theta) is listed with I(rel)=100 as the most intensive peak. However, in the graphical representation of the diffractogram the broad reflex in the range of 20.5-20.7 (2 Theta) appears to be the reflex with the highest intensity. The reason for this is the overlap of the reflexes at 20.56; 20.60, 20.61 and 20.73 (2 Theta).

The uncertainty in the 2Θ values is ±0.2° in 2Θ. The link between the d-values and the 2Theta values are known from the Bragg equation, which is well known to any person skilled in the art:

2*d*sin(Theta)=n*lamda

wherein

d: d-value

Theta: incident angle Theta

n: integral number (n=1, 2, 3, . . . )

lambda: Wave length (CuKα (1.540598))

### Comparison Between Calculated and Measured Diffractogram:

For reasons of comparison with the calculated diffractogram of form A (FIG. 1) an experimentally measured diffractogram (FIG. 2) is analysed with regard to the peak positions.

### Parameter of Measurement. (Device: STOE, Stadi P):

### Diffractometer: Transmission

### Monochromator: Curved Germanium(111)

### Wavelength: 1.540598 Cu

### Detector: Linear PSD

Scan Mode Transmission/Moving PSD/Fixed omega

### Scan Type: 2Theta:Omega

The peak positions of the measured diffractograms were determined using the program WinXPow from the Stoe company. The parameters used for peak-search were:

Expected halfwidth: 0.150

Significance level: 2.5

Peak height level: 50

The peak lists were checked and processed manually.

The calculated diffractogram and the measured diffractogram are in very good agreement (FIG. 3.)

### Crystalline Modification A

(a) The following Table 3 shows the peak list for crystalline modification A as measured for the product obtained according to example 1.2. The uncertainty in the 2Θ values is ±0.2° in 2Θ; rel. I (or RI) is the relative intensity of the respective peaks. Maximum intensity is 100.

TABLE 3

| 2Θ | rel. I | 2Θ | rel. I | 2Θ | rel. I | 2Θ | rel. I | 2Θ | rel. I |
|---|---|---|---|---|---|---|---|---|---|
| 10.75 | 27 | 12.89 | 47 | 13.35 | 1 | 13.95 | 45 | 16.78 | 71 |
| 17.55 | 3 | 18.37 | 19 | 18.88 | 17 | 19.63 | 29 | 20.64 | 100 |
| 21.12 | 3 | 21.53 | 29 | 22.37 | 1 | 22.68 | 16 | 23.29 | 2 |
| 23.49 | 4 | 23.90 | 15 | 24.92 | 9 | 25.18 | 3 | 25.84 | 2 |
| 26.42 | 14 | 27.89 | 3 | 28.69 | 3 | 29.48 | 3 | 30.14 | 4 |
| 30.51 | 4 | 31.74 | 1 | 32.36 | 3 | 33.10 | 6 | 33.78 | 3 |
| 34.40 | 3 | 35.45 | 2 | 35.94 | 2 | 36.60 | 2 | 36.76 | 1 |
| 37.07 | 4 | 38.10 | 1 | 38.41 | 4 | 39.43 | 3 | 40.38 | 4 |
| 40.82 | 4 | 41.74 | 2 | 42.32 | 2 | 42.66 | 3 | 43.77 | 1 |
| 44.78 | 1 | 45.19 | 2 | 46.69 | 1 | 47.41 | 3 | 47.99 | 1 |
| 49.04 | 2 | | | | | | | | |

(b) The following Table 4 shows the peak list for crystalline modification A as calculated from the single crystal data for the product obtained according to example 4-1. The uncertainty in the 2Θ values is ±0.2° in 2Θ; rel. I (or RI) is the relative intensity of the respective peaks. Maximum intensity is 100.

TABLE 4

| 2Θ | rel. I | 2Θ | rel. I | 2Θ | rel. I | 2Θ | rel. I | 2Θ | rel. I |
|---|---|---|---|---|---|---|---|---|---|
| 10.24 | 1 | 10.69 | 55 | 10.77 | 1 | 12.81 | 42 | 13.82 | 15 |
| 13.88 | 53 | 14.22 | 1 | 16.71 | 100 | 17.52 | 3 | 18.31 | 18 |
| 18.32 | 8 | 18.62 | 0 | 18.76 | 20 | 19.52 | 23 | 19.89 | 2 |
| 20.56 | 60 | 20.60 | 71 | 20.61 | 15 | 20.73 | 0 | 21.42 | 33 |
| 21.48 | 4 | 21.64 | 3 | 22.63 | 21 | 23.22 | 2 | 23.37 | 4 |
| 23.47 | 0 | 23.85 | 17 | 24.79 | 10 | 25.08 | 6 | 25.67 | 2 |
| 25.77 | 1 | 26.33 | 3 | 26.34 | 15 | 26.96 | 0 | 27.85 | 4 |
| 27.97 | 0 | 28.07 | 0 | 28.37 | 0 | 28.56 | 0 | 28.57 | 0 |
| 28.59 | 2 | 28.66 | 5 | 28.82 | 1 | 29.43 | 4 | 29.67 | 1 |
| 29.93 | 4 | 30.02 | 0 | 30.11 | 4 | 30.17 | 1 | 30.33 | 5 |
| 30.52 | 4 | 31.06 | 0 | 31.62 | 1 | 32.31 | 3 | 32.46 | 3 |
| 32.51 | 0 | 32.59 | 1 | 32.71 | 1 | 33.03 | 0 | 33.05 | 6 |
| 33.48 | 0 | 33.67 | 2 | 33.71 | 2 | 33.79 | 2 | 33.92 | 1 |
| 33.92 | 1 | 34.23 | 3 | 34.28 | 1 | 34.39 | 4 | 35.02 | 2 |
| 35.24 | 1 | 35.46 | 2 | 35.61 | 1 | 35.80 | 3 | 35.89 | 0 |
| 36.53 | 2 | 36.75 | 1 | 36.92 | 4 | 37.11 | 0 | 37.14 | 2 |
| 37.16 | 1 | 37.34 | 1 | 37.62 | 0 | 38.08 | 2 | 38.36 | 5 |
| 38.38 | 1 | 38.45 | 1 | 38.82 | 1 | 39.11 | 0 | 39.28 | 0 |
| 39.29 | 1 | 39.36 | 3 | 39.47 | 2 | 39.48 | 0 | 39.50 | 0 |
| 39.63 | 1 | | | | | | | | |

### Crystalline Modification B

The following Table 5 shows the peak list for crystalline modification B as calculated from the single crystal data for the product obtained according to example 4-5. The uncertainty in the 2Θ values is ±0.2° in 2Θ; rel. I (or RI) is the relative intensity of the respective peaks. Maximum intensity is 100.

TABLE 5

| 2Θ | rel. I | 2Θ | rel. I | 2Θ | rel. I | 2Θ | rel. I | 2Θ | rel. I |
|---|---|---|---|---|---|---|---|---|---|
| 9.72 | 4 | 11.35 | 39 | 12.75 | 68 | 12.79 | 2 | 14.04 | 22 |
| 15.23 | 7 | 16.51 | 15 | 18.79 | 100 | 19.20 | 10 | 19.50 | 0 |
| 19.74 | 30 | 20.09 | 53 | 20.94 | 0 | 21.20 | 20 | 21.42 | 6 |
| 22.24 | 0 | 22.82 | 3 | 23.69 | 14 | 23.76 | 9 | 24.30 | 5 |
| 25.19 | 0 | 25.66 | 9 | 25.74 | 10 | 25.84 | 5 | 26.55 | 3 |
| 26.77 | 2 | 27.07 | 2 | 27.23 | 0 | 27.42 | 0 | 27.83 | 1 |
| 28.07 | 1 | 28.30 | 12 | 28.49 | 2 | 28.52 | 0 | 29.44 | 2 |
| 29.74 | 3 | 30.21 | 1 | 30.27 | 1 | 30.62 | 1 | 30.74 | 3 |
| 31.81 | 6 | 31.96 | 1 | 32.01 | 3 | 32.09 | 3 | 33.14 | 0 |
| 33.18 | 1 | 33.39 | 2 | 33.62 | 0 | 33.94 | 1 | 34.01 | 1 |
| 34.11 | 0 | 34.25 | 2 | 34.51 | 0 | 34.52 | 1 | 34.85 | 2 |
| 35.15 | 0 | 35.37 | 2 | 35.54 | 0 | 35.55 | 1 | 35.70 | 4 |
| 35.83 | 2 | 35.89 | 0 | 36.11 | 0 | 36.30 | 0 | 37.21 | 1 |
| 37.29 | 2 | 37.63 | 3 | 38.06 | 0 | 38.12 | 1 | 38.20 | 1 |
| 38.48 | 1 | 38.50 | 2 | 38.75 | 0 | 38.91 | 0 | 38.96 | 1 |
| 39.01 | 0 | 39.04 | 2 | 39.08 | 0 | 39.47 | 3 | 39.60 | 0 |
| 39.97 | 2 | | | | | | | | |

### Crystalline Modification C

The following table 6 shows the peak list for crystalline modification C as measured for the product obtained according to example 3. The uncertainty in the 2Θ values is ±0.2° in 2Θ; rel. I (or RI) is the relative intensity of the respective peaks. Maximum intensity is 100.

TABLE 6

| 2Θ | rel. I | 2Θ | rel. I | 2Θ | rel. I | 2Θ | rel. I | 2Θ | rel. I |
|---|---|---|---|---|---|---|---|---|---|
| 9.05 | 4 | 10.04 | 4 | 12.54 | 5 | 14.64 | 100 | 15.02 | 12 |
| 15.47 | 22 | 15.83 | 29 | 16.07 | 8 | 16.84 | 15 | 17.77 | 5 |
| 18.07 | 61 | 19.64 | 42 | 20.23 | 28 | 21.04 | 20 | 21.49 | 15 |
| 22.04 | 61 | 23.78 | 3 | 24.79 | 35 | 24.94 | 12 | 25.18 | 12 |
| 25.69 | 47 | 25.82 | 14 | 26.34 | 10 | 26.82 | 5 | 27.80 | 15 |
| 28.22 | 39 | 29.25 | 12 | 29.46 | 9 | 29.89 | 10 | 30.07 | 8 |
| 30.31 | 3 | 30.64 | 4 | 31.17 | 18 | 32.47 | 4 | 32.94 | 3 |
| 33.21 | 4 | 34.00 | 5 | 34.40 | 4 | 35.90 | 5 | 36.34 | 5 |
| 38.13 | 2 | 39.12 | 5 | 39.31 | 3 | | | | |

### Analysis—X-Ray Single Crystal Structure Determination

X-ray single crystal diffraction analyses of the modification A (tables 7 to 11) and B (tables 12 to 16) were performed with STOE IPDS.

radiation source: fine-focus sealed tube

radiation type: Mo Kα

wavelength: 0.71073 Å

measurement method: Phi-rotation

Tube power [kW]: 1.00

Tube voltage [kV]: 50

Tube current [mA]: 20

Collimator size [mm]: 0.3

Temperature [K]: 293

### Software:

Data collection: STOE IPDS-EXPOSE Vers. 2.87 (1997)
Cell refinement: STOE IPDS-RECIPE/CELL Vers. 287 (1997)
Data reduction: STOE IPDS-PROFILE/INTEGRATE Vers.2.87 (1997)
Structure solution: SHELXS-86 (Sheldrick, 1990)
SIR97 (Cascarano al., Acta Cryst., 1996, A52, C-79)
   Structure refinement: SHELXL-93 (Sheldrick, 1993)

X-ray single crystal diffraction analyses for determination of the absolute configuration were either performed with NONIUS CAD4 using radiation type Fe Kα wavelength 1.93604 Å, at 291±2 K, graphite monochromator, and/or Bruker D8 Goniometer with SMART-APEX-detector, Mo Kα, at 100 K.

### Software:

Data collection: CAD4_(Enraf-Nonius,_{—}1977)
Cell refinement: CAD4_(Enraf-Nonius,_{—}1977)
Data reduction: PROCESS_MoIEN_(Fair,_{—}1990)
Structure solution: SHELXS-97 (Sheldrick, 1990)
Structure refinement: SHELXL-97 (Sheldrick, 1997)

The correct assignment of the absolute configuration was proved by calculation of the Flack-Parameter and/or analysis of Bijvoet differences by means of Bayesian statistics (Platon 2006)

### Crystalline Modification A:

TABLE 7
Crystal data and structure refinement for crystalline
modification A obtained according to example 4-1

| Empirical formula | C16 H25 N O3 | |
|---|---|---|
| Formula weight | 279.37 | |
| Temperature | 293(2) K | |
| Wavelength | 0.71073 Å | |
| Crystal system, space group | 1st, P212121 | |
| Unit cell dimensions | a = 9.453 (2) Å | alpha = 90 deg. |
| | b = 10.118 (2) Å | beta = 90 deg. |
| | c = 16.538 (3) Å | gamma = 90 deg. |

| | |
|---|---|
| Volume | 1581.8(5) Å\hat{ }3 |
| Z, Calculated density | 4, 1.173 Mg/m\hat{ }3 |
| Absorption coefficient | 0.080 mm\hat{ }−1 |
| F(000) | 608 |
| Theta range for data collection | 2.36 to 24.15 deg. |
| Limiting indices | −10 <= h <= 0, −11 <= k <= 11, −18 <= l <= 18 |
| Reflections collected/unique | 12014/2424 [R(int) = 0.0904] |
| Absorption correction | No correction |
| Refinement method | Full-matrix least-squares on F\hat{ }2 |
| Data/restraints/parameters | 2424/0/282 |
| Goodness-of-fit on F\hat{ }2 | 0.635 |
| Final R indices [I > 2sigma (I)] | R1 = 0.0373, wR2 = 0.0867 |
| R indices (all data) | R1 = 0.0511, wR2 = 0.0948 |
| Absolute structure parameter | −0.22(141) |
| Extinction coefficient | 0.0132(29) |
| Largest diff. peak and hole | 0.137 and −0.180 e.Å\hat{ }−3 |

TABLE 8
Atomic coordinates (×10\hat{ }4) and equivalent isotropic displacement
parameters (Å\hat{ }2 × 10\hat{ }3).

| | | | |
|---|---|---|---|
| x | y | z | U(eq) |

| | | | | | | | |
|---|---|---|---|---|---|---|---|
| C(1) | −437 | (2) | 265 | (2) | −1844 | (1) | 39 (1) |
| C(2) | −98 | (3) | 1204 | (2) | −1256 | (1) | 44 (1) |
| C(3) | 1246 | (3) | 1248 | (3) | −930 | (1) | 51 (1) |
| C(4) | 2288 | (3) | 400 | (3) | −1198 | (2) | 61 (1) |
| C(5) | 1954 | (3) | −542 | (3) | −1769 | (2) | 58 (1) |
| C(6) | 605 | (2) | −610 | (2) | −2088 | (1) | 48 (1) |
| C(7) | −1914 | (2) | 236 | (2) | −2221 | (1) | 39 (1) |
| C(8) | −2648 | (2) | −1114 | (2) | −2123 | (1) | 42 (1) |
| C(9) | −4061 | (3) | −1146 | (3) | −2570 | (1) | 54 (1) |
| C(10) | −3920 | (3) | −765 | (3) | −3463 | (1) | 52 (1) |
| C(11) | −3188 | (3) | 534 | (2) | −3576 | (1) | 49 (1) |
| C(12) | −1777 | (2) | 563 | (3) | −3134 | (1) | 44 (1) |
| C(13) | −2847 | (3) | −1447 | (3) | −1222 | (1) | 51 (1) |
| C(14) | 643 | (4) | 3014 | (4) | −30 | (2) | 71 (1) |
| C(15) | −3761 | (5) | −2873 | (5) | −193 | (2) | 81 (1) |
| C(16) | −2330 | (4) | −3782 | (4) | −1245 | (2) | 80 (1) |
| O(1) | 1655 | (2) | 2116 | (2) | −333 | (1) | 76 (1) |
| O(2) | −2744 | (2) | 1231 | (2) | −1832 | (1) | 52 (1) |
| O(3) | −3989 | (2) | 1616 | (2) | −3269 | (1) | 65 (1) |
| N(1) | −3381 | (2) | −2770 | (2) | −1055 | (1) | 50 (1) |

U(eq) is defined as one third of the trace of the orthogonalized Uij tensor.

TABLE 9
Bond lengths [Å] and angles [deg]:

| | | |
|---|---|---|
| C(1)-C(6) | 1.384 | (3) |
| C(1)-C(2) | 1.397 | (3) |
| C(1)-C(7) | 1.528 | (3) |
| C(2)-C(3) | 1.381 | (3) |
| C(3)-O(1) | 1.377 | (3) |
| C(3)-C(4) | 1.380 | (4) |
| C(4)-C(5) | 1.379 | (4) |
| C(5)-C(6) | 1.381 | (3) |
| C(7)-O(2) | 1.430 | (2) |
| C(7)-C(8) | 1.541 | (3) |
| C(7)-C(12) | 1.552 | (3) |
| C(8)-C(9) | 1.526 | (3) |
| C(8)-C(13) | 1.540 | (3) |
| C(9)-C(10) | 1.532 | (3) |
| C(10)-C(11) | 1.497 | (4) |
| C(11)-O(3) | 1.425 | (3) |
| C(11)-C(12) | 1.522 | (3) |
| C(13)-N(1) | 1.457 | (3) |
| C(14)-O(1) | 1.411 | (4) |
| C(15)-N(1) | 1.475 | (3) |
| C(16)-N(1) | 1.461 | (4) |
| C(6)-C(1)-C(2) | 118.3 | (2) |
| C(6)-C(1)-C(7) | 121.3 | (2) |
| C(2)-C(1)-C(7) | 120.4 | (2) |
| C(3)-C(2)-C(1) | 120.4 | (2) |
| O(1)-C(3)-C(4) | 115.3 | (2) |
| O(1)-C(3)-C(2) | 124.0 | (2) |
| C(4)-C(3)-C(2) | 120.7 | (2) |
| C(5)-C(4)-C(3) | 119.1 | (2) |
| C(4)-C(5)-C(6) | 120.4 | (2) |
| C(5)-C(6)-C(1) | 121.0 | (2) |
| O(2)-C(7)-C(1) | 107.72 | (15) |
| O(2)-C(7)-C(8) | 109.2 | (2) |
| C(1)-C(7)-C(8) | 112.7 | (2) |
| O(2)-C(7)-C(12) | 109.5 | (2) |
| C(1)-C(7)-C(12) | 108.4 | (2) |
| C(8)-C(7)-C(12) | 109.2 | (2) |
| C(9)-C(8)-C(13) | 110.9 | (2) |
| C(9)-C(8)-C(7) | 111.2 | (2) |
| C(13)-C(8)-C(7) | 110.5 | (2) |
| C(8)-C(9)-C(10) | 112.7 | (2) |
| C(11)-C(10)-C(9) | 112.5 | (2) |
| O(3)-C(11)-C(10) | 112.6 | (2) |
| O(3)-C(11)-C(12) | 106.2 | (2) |
| C(10)-C(11)-C(12) | 111.2 | (2) |
| C(11)-C(12)-C(7) | 113.0 | (2) |
| N(1)-C(13)-C(8) | 115.2 | (2) |
| C(3)-O(1)-C(14) | 118.3 | (2) |
| C(13)-N(1)-C(16) | 111.5 | (2) |
| C(13)-N(1)-C(15) | 109.4 | (2) |
| C(16)-N(1)-C(15) | 108.9 | (3) |

### Symmetry Transformations Used to Generate Equivalent Atoms:

TABLE 10
Anisotropic displacement parameters (Å\hat{ }2 × 10\hat{ }3)

| | U11 | U22 | U33 | U23 | | U13 | | U12 | |
|---|---|---|---|---|---|---|---|---|---|
| C(1) | 39 (1) | 38 (1) | 42 (1) | 2 | (1) | 2 | (1) | −2 | (1) |
| C(2) | 46 (1) | 43 (1) | 43 (1) | 0 | (1) | 2 | (1) | −5 | (1) |
| C(3) | 48 (1) | 58 (2) | 47 (1) | 5 | (1) | −8 | (1) | −14 | (1) |
| C(4) | 45 (2) | 73 (2) | 64 (1) | 11 | (1) | −15 | (1) | −10 | (1) |
| C(5) | 41 (1) | 59 (2) | 74 (2) | 9 | (1) | 1 | (1) | 9 | (1) |
| C(6) | 45 (1) | 45 (1) | 55 (1) | −1 | (1) | 2 | (1) | 3 | (1) |
| C(7) | 36 (1) | 41 (1) | 39 (1) | −9 | (1) | 4 | (1) | 1 | (1) |
| C(8) | 42 (1) | 50 (1) | 35 (1) | −7 | (1) | 5 | (1) | −2 | (1) |
| C(9) | 46 (1) | 71 (2) | 45 (1) | −4 | (1) | −2 | (1) | −18 | (1) |
| C(10) | 48 (1) | 63 (2) | 44 (1) | −12 | (1) | −8 | (1) | −1 | (1) |
| C(11) | 50 (1) | 54 (1) | 44 (1) | −4 | (1) | −1 | (1) | 5 | (1) |
| C(12) | 42 (1) | 48 (1) | 42 (1) | 1 | (1) | 3 | (1) | 5 | (1) |
| C(13) | 55 (2) | 62 (2) | 36 (1) | −5 | (1) | 3 | (1) | −14 | (1) |
| C(14) | 89 (2) | 74 (2) | 49 (1) | −10 | (2) | −6 | (2) | −19 | (2) |
| C(15) | 99 (3) | 99 (3) | 44 (1) | 14 | (2) | 3 | (2) | −32 | (3) |
| C(16) | 70 (2) | 73 (2) | 97 (2) | 9 | (2) | −4 | (2) | 11 | (2) |
| O(1) | 69 (1) | 92 (2) | 68 (1) | −21 | (1) | −17 | (1) | −20 | (1) |
| O(2) | 43 (1) | 60 (1) | 54 (1) | −23 | (1) | −2 | (1) | 12 | (1) |
| O(3) | 54 (1) | 59 (1) | 81 (1) | −19 | (1) | −23 | (1) | 16 | (1) |
| N(1) | 51 (1) | 59 (1) | 41 (1) | 2 | (1) | −1 | (1) | −8 | (1) |

The anisotropic displacement factor exponent takes the form: −2 pi\hat{ }2 [h\hat{ }2 a*\hat{ }2 U11 + . . . + 2 h k a* b* U12]

TABLE 11
Hydrogen coordinates (×10\hat{ }4) and isotropic displacement parameters
(Å\hat{ }2 × 10\hat{ }3).

| | x | | y | | z | | U(eq) | |
|---|---|---|---|---|---|---|---|---|
| H(2) | −824 | (27) | 1813 | (26) | −1117 | (14) | 56 | (7) |
| H(4) | 3288 | (27) | 506 | (23) | −990 | (14) | 52 | (6) |
| H(5) | 2616 | (27) | −1179 | (27) | −1906 | (16) | 65 | (7) |
| H(6) | 351 | (27) | −1345 | (30) | −2468 | (17) | 66 | (7) |
| H(8) | −1994 | (24) | −1796 | (23) | −2345 | (13) | 50 | (6) |
| H(91) | −5501 | (27) | −7060 | (30) | −2504 | (13) | 60 | (7) |
| H(92) | −4817 | (27) | −531 | (26) | −2294 | (13) | 55 | (6) |
| H(101) | −3282 | (28) | −1476 | (27) | −3738 | (15) | 68 | (7) |
| H(102) | −4892 | (28) | −858 | (22) | −3679 | (13) | 49 | (6) |
| H(11) | −3009 | (28) | 670 | (26) | −4162 | (17) | 77 | (8) |
| H(121) | −1321 | (22) | 1465 | (25) | −3176 | (14) | 45 | (6) |
| H(122) | −1165 | (22) | −161 | (23) | −3409 | (12) | 42 | (5) |
| H(131) | −3582 | (29) | −759 | (26) | −1022 | (16) | 67 | (8) |
| H(132) | −1958 | (30) | −1361 | (25) | −949 | (15) | 63 | (7) |
| H(141) | −234 | (42) | 2535 | (46) | 153 | (21) | 121 | (13) |
| H(142) | 359 | (29) | 3658 | (29) | −423 | (18) | 75 | (9) |
| H(143) | 1223 | (37) | 3480 | (38) | 408 | (21) | 110 | (12) |
| H(151) | −4207 | (29) | −3709 | (33) | −103 | (17) | 73 | (8) |
| H(152) | −2902 | (36) | −2772 | (34) | 140 | (20) | 92 | (10) |
| H(153) | −4571 | (40) | −2318 | (44) | −104 | (22) | 106 | (14) |
| H(161) | −2722 | (40) | −4638 | (39) | −1053 | (22) | 114 | (12) |
| H(162) | −1420 | (45) | −3575 | (40) | −968 | (24) | 118 | (13) |
| H(163) | −1938 | (40) | −3750 | (39) | −1790 | (27) | 115 | (12) |
| H(20) | −3362 | (32) | 1599 | (29) | −2135 | (17) | 71 | (9) |
| H(30) | −4942 | (35) | 1642 | (30) | −3482 | (17) | 82 | (9) |

### Crystalline Modification B:

TABLE 12
Crystal data and structure refinement for crystalline
modification B obtained according to example 4-5

| Empirical formula | C16 H25 N O3 | |
|---|---|---|
| Formula weight | 279.37 | |
| Temperature | 293(2) K | |
| Wavelength | 0.71073 Å | |
| Crystal system, space group | 1st, P21 | |
| Unit cell dimensions | a = 8.0920 (9) Å | alpha = 90 deg. |
| | b = 10.727 (2) Å | beta = 105.751(13) deg. |
| | c = 9.4510 (10) Å | gamma = 90 deg. |

| | |
|---|---|
| Volume | 789.6(2) Å\hat{ }3 |
| Z, Calculated density | 2, 1.175 Mg/m\hat{ }3 |
| Absorption coefficient | 0.080 mm\hat{ }−1 |
| F(000) | 304 |
| Theta range for data collection | 2.24 to 23.85 deg. |
| Limiting indices | −9 <= h <= 9, −12 <= k <= 12, −10 <= l <= 10 |
| Reflections collected/unique | 8316/2360 [R(int) = 0.0702] |
| Absorption correction | No correction |
| Refinement method | Full-matrix least-squares on F\hat{ }2 |
| Data/restraints/parameters | 2360/1/281 |
| Goodness-of-fit on F\hat{ }2 | 0.651 |
| Final R indices [I > 2sigma (I)] | R1 = 0.0296, wR2 = 0.0781 |
| R indices (all data) | R1 = 0.0334, wR2 = 0.0817 |
| Absolute structure parameter | −0.72(102) |
| Largest diff. peak and hole | 0.137 and −0.121 e.Å\hat{ }−3 |

TABLE 13
Atomic coordinates (×10\hat{ }4) and equivalent isotropic displacement
parameters (Å\hat{ }2 × 10\hat{ }3).

| | | | |
|---|---|---|---|
| x | y | z | U(eq) |

| | | | | | | | |
|---|---|---|---|---|---|---|---|
| C(1) | 3732 | (2) | 989 | (1) | 9252 | (2) | 34 (1) |
| C(2) | 2593 | (2) | 1904 | (2) | 9434 | (2) | 38 (1) |
| C(3) | 2039 | (2) | 1946 | (2) | 10701 | (2) | 46 (1) |
| C(4) | 2623 | (3) | 1071 | (2) | 11804 | (2) | 54 (1) |
| C(5) | 3736 | (3) | 154 | (2) | 11625 | (2) | 49 (1) |
| C(6) | 4290 | (2) | 104 | (2) | 10365 | (2) | 41 (1) |
| C(7) | 4435 | (2) | 1013 | (1) | 7909 | (2) | 33 (1) |
| C(8) | 4248 | (2) | −259 | (2) | 7101 | (2) | 37 (1) |
| C(9) | 5129 | (3) | −219 | (2) | 5866 | (2) | 48 (1) |
| C(10) | 7002 | (2) | 174 | (2) | 6386 | (2) | 45 (1) |
| C(11) | 7222 | (2) | 1409 | (2) | 7191 | (2) | 44 (1) |
| C(12) | 6350 | (2) | 1382 | (2) | 8425 | (2) | 38 (1) |
| C(13) | 2352 | (2) | −629 | (2) | 6525 | (2) | 45 (1) |
| C(14) | 248 | (3) | 3720 | (2) | 9889 | (3) | 62 (1) |
| C(15) | 2534 | (4) | −2818 | (2) | 7195 | (3) | 66 (1) |
| C(16) | 235 | (3) | −2093 | (3) | 5205 | (3) | 70 (1) |
| O(1) | 927 | (2) | 2817 | (2) | 10968 | (2) | 71 (1) |
| O(2) | 3493 | (2) | 1941 | (1) | 6921 | (1) | 46 (1) |
| O(3) | 6467 | (2) | 2425 | (1) | 6261 | (2) | 59 (1) |
| N(1) | 2065 | (2) | −1922 | (1) | 5988 | (2) | 45 (1) |

U(eq) is defined as one third of the trace of the orthogonalized Uij tensor.

TABLE 14
Bond lengths [Å] and angles [deg].

| | | |
|---|---|---|
| C(1)-C(2) | 1.388 | (2) |
| C(1)-C(6) | 1.398 | (2) |
| C(1)-C(7) | 1.526 | (2) |
| C(2)-C(3) | 1.388 | (2) |
| C(3)-O(1) | 1.367 | (2) |
| C(3)-C(4) | 1.388 | (3) |
| C(4)-C(5) | 1.374 | (3) |
| C(5)-C(6) | 1.383 | (3) |
| C(7)-O(2) | 1.434 | (2) |
| C(7)-C(12) | 1.545 | (2) |
| C(7)-C(8) | 1.550 | (2) |
| C(8)-C(9) | 1.525 | (2) |
| C(8)-C(13) | 1.535 | (2) |
| C(9)-C(10) | 1.520 | (3) |
| C(10)-C(11) | 1.513 | (3) |
| C(11)-O(3) | 1.429 | (2) |
| C(11)-C(12) | 1.519 | (2) |
| C(13)-N(1) | 1.473 | (2) |
| C(14)-O(1) | 1.406 | (3) |
| C(15)-N(1) | 1.461 | (3) |
| C(16)-N(1) | 1.477 | (3) |
| C(2)-C(1)-C(6) | 118.5 | (2) |
| C(2)-C(1)-C(7) | 120.07 | (13) |
| C(6)-C(1)-C(7) | 121.29 | (14) |
| C(1)-C(2)-C(3) | 120.7 | (2) |
| O(1)-C(3)-C(4) | 115.5 | (2) |
| O(1)-C(3)-C(2) | 124.4 | (2) |
| C(4)-C(3)-C(2) | 120.2 | (2) |
| C(5)-C(4)-C(3) | 119.5 | (2) |
| C(4)-C(5)-C(6) | 120.7 | (2) |
| C(5)-C(6)-C(1) | 120.4 | (2) |
| O(2)-C(7)-C(1) | 107.49 | (12) |
| O(2)-C(7)-C(12) | 109.75 | (12) |
| C(1)-C(7)-C(12) | 108.04 | (12) |
| O(2)-C(7)-C(8) | 109.09 | (12) |
| C(1)-C(7)-C(8) | 112.76 | (12) |
| C(12)-C(7)-C(8) | 109.66 | (13) |
| C(9)-C(8)-C(13) | 111.44 | (14) |
| C(9)-C(8)-C(7) | 110.42 | (14) |
| C(13)-C(8)-C(7) | 110.90 | (13) |
| C(10)-C(9)-C(8) | 113.07 | (14) |
| C(11)-C(10)-C(9) | 112.1 | (2) |
| O(3)-C(11)-C(10) | 112.70 | (14) |
| O(3)-C(11)-C(12) | 106.05 | (14) |
| C(10)-C(11)-C(12) | 110.69 | (15) |
| C(11)-C(12)-C(7) | 113.42 | (13) |
| N(1)-C(13)-C(8) | 114.51 | (14) |
| C(3)-O(1)-C(14) | 118.9 | (2) |
| C(15)-N(1)-C(13) | 111.54 | (15) |
| C(15)-N(1)-C(16) | 108.8 | (2) |
| C(13)-N(1)-C(16) | 109.4 | (2) |

### Symmetry Transformations Used to Generate Equivalent Atoms:

TABLE 15
Anisotropic displacement parameters (Å\hat{ }2 × 10\hat{ }3).

| | U11 | U22 | U33 | U23 | | U13 | | U12 | |
|---|---|---|---|---|---|---|---|---|---|
| C(1) | 38 (1) | 35 (1) | 28 (1) | −1 | (1) | 7 | (1) | −3 | (1) |
| C(2) | 41 (1) | 41 (1) | 31 (1) | −3 | (1) | 10 | (1) | 0 | (1) |
| C(3) | 45 (1) | 53 (1) | 43 (1) | −10 | (1) | 17 | (1) | −1 | (1) |
| C(4) | 65 (1) | 66 (1) | 36 (1) | −3 | (1) | 22 | (1) | −6 | (1) |
| C(5) | 62 (1) | 52 (1) | 33 (1) | 6 | (1) | 14 | (1) | −4 | (1) |
| C(6) | 48 (1) | 41 (1) | 35 (1) | 1 | (1) | 12 | (1) | 2 | (1) |
| C(7) | 38 (1) | 36 (1) | 27 (1) | 4 | (1) | 9 | (1) | 7 | (1) |
| C(8) | 35 (1) | 44 (1) | 31 (1) | −1 | (1) | 6 | (1) | 7 | (1) |
| C(9) | 49 (1) | 60 (1) | 37 (1) | −11 | (1) | 16 | (1) | 3 | (1) |
| C(10) | 44 (1) | 50 (1) | 46 (1) | 1 | (1) | 21 | (1) | 8 | (1) |
| C(11) | 45 (1) | 44 (1) | 46 (1) | 4 | (1) | 18 | (1) | 2 | (1) |
| C(12) | 41 (1) | 39 (1) | 34 (1) | 3 | (1) | 10 | (1) | 0 | (1) |
| C(13) | 38 (1) | 54 (1) | 43 (1) | −13 | (1) | 9 | (1) | 4 | (1) |
| C(14) | 53 (1) | 65 (1) | 67 (1) | −19 | (1) | 12 | (1) | 11 | (1) |
| C(15) | 78 (2) | 59 (1) | 65 (1) | 4 | (1) | 26 | (1) | −9 | (1) |
| C(16) | 42 (1) | 85 (2) | 79 (2) | −32 | (2) | 11 | (1) | −11 | (1) |
| O(1) | 77 (1) | 84 (1) | 61 (1) | −7 | (1) | 35 | (1) | 25 | (1) |
| O(2) | 51 (1) | 53 (1) | 34 (1) | 15 | (1) | 13 | (1) | 21 | (1) |
| O(3) | 80 (1) | 51 (1) | 62 (1) | 22 | (1) | 45 | (1) | 19 | (1) |
| N(1) | 36 (1) | 57 (1) | 42 (1) | −14 | (1) | 12 | (1) | −5 | (1) |

The anisotropic displacement factor exponent takes the form: −2 pi\hat{ }2 [h\hat{ }2 a*\hat{ }2 U11 + . . . + 2 h k a* b* U12]

TABLE 16
Hydrogen coordinates (×10\hat{ }4) and isotropic displacement parameters
(Å\hat{ }2 × 10\hat{ }3).

| | x | | y | | z | | U(eq) | |
|---|---|---|---|---|---|---|---|---|
| H(2) | 2292 | (26) | 2489 | (19) | 8728 | (23) | 47 | (5) |
| H(4) | 2267 | (31) | 1123 | (20) | 12698 | (26) | 63 | (6) |
| H(5) | 4091 | (29) | −504 | (24) | 12298 | (26) | 65 | (6) |
| H(6) | 5086 | (26) | −504 | (19) | 10213 | (20) | 39 | (4) |
| H(8) | 4801 | (24) | −870 | (18) | 7773 | (21) | 38 | (4) |
| H(91) | 5047 | (26) | −1089 | (20) | 5429 | (21) | 49 | (5) |
| H(92) | 4480 | (27) | 346 | (21) | 5076 | (26) | 55 | (6) |
| H(101) | 7458 | (31) | 231 | (21) | 5497 | (27) | 66 | (6) |
| H(102) | 7605 | (26) | −526 | (20) | 7008 | (22) | 46 | (5) |
| H(11) | 8509 | (26) | 1586 | (16) | 7589 | (20) | 46 | (5) |
| H(121) | 6453 | (24) | 2258 | (20) | 8843 | (20) | 44 | (5) |
| H(122) | 6960 | (24) | 711 | (17) | 9220 | (22) | 42 | (5) |
| H(131) | 1865 | (27) | −42 | (19) | 5691 | (23) | 51 | (5) |
| H(132) | 1713 | (27) | −484 | (19) | 7304 | (24) | 53 | (5) |
| H(141) | −391 | (37) | 4244 | (28) | 10305 | (31) | 83 | (8) |
| H(142) | 1107 | (37) | 4219 | (25) | 9546 | (27) | 77 | (7) |
| H(143) | −338 | (33) | 3380 | (25) | 8943 | (27) | 76 | (7) |
| H(151) | 2213 | (35) | −3760 | (26) | 6838 | (28) | 81 | (7) |
| H(152) | 3796 | (47) | −2729 | (27) | 7747 | (35) | 104 | (10) |
| H(153) | 1861 | (40) | −2763 | (26) | 7862 | (32) | 91 | (9) |
| H(161) | 41 | (44) | −3006 | (33) | 4807 | (36) | 102 | (9) |
| H(162) | −573 | (36) | −1888 | (25) | 5813 | (28) | 80 | (7) |
| H(163) | 81 | (31) | −1512 | (24) | 4389 | (28) | 64 | (7) |
| H(30) | 7064 | (36) | 2493 | (26) | 5514 | (31) | 86 | (8) |
| H(20) | 4176 | (42) | 2297 | (32) | 6436 | (36) | 103 | (10) |

### Analysis—DSC

Differential Scanning calorimetry (DSC): device reference Mettler Toledo DSC 821, perforated 40 μm aluminium standard crucible, variable temperature range and variable heating-rate, nitrogen atmosphere. Unless otherwise stated, substance quantities within the range from 2 mg to 20 mg were employed.

The measurement took place in a nitrogen flow in a temperature range from 30±5° C. to 200° C. with a heating rate of 10° C./min. The temperatures specified in relation to DSC analyses are, unless otherwise specified, the temperatures of the peak maxima (peak temperature T_{P}). Onset temperatures of peaks are indicated by T_{O}.

### Crystalline Modification A According to Example 1.2:

T_{O} 115.96° C.; T_{P} 118.53° C.; J/g 126.60.

### Crystalline Modification B According to Example 2:

T_{O} 109.82° C.; T_{P} 114.12° C.; J/g 168.54.

### Crystalline Modification C According to Example 3:

T_{O} 115.66° C.; T_{P} 118.72° C.; J/g 111.54.

Analysis—FT Raman spectroscopy

Crystalline modification A was characterized by means of Fourier transform (FT) Raman spectrometry. For this purpose, the FT Raman spectra were recorded on a Bruker RFS100 Raman spectrometer (Nd-YAG 100 mW laser, excitation 1064 nm, Ge detector, 64 scans, 25-3500 cm⁻¹, resolution 4 cm⁻¹).

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A crystalline modification of (1R,3R,6R)-6-dimethylaminomethyl-1-(3-methoxyphenyl)-cyclohexane-1,3-diol, or (1S,3S,6S)-6-dimethylaminomethyl-1-(3-methoxyphenyl)-cyclohexane-1,3-diol, or a mixture thereof.

2. A crystalline modification according to claim 1, wherein said crystalline modification is crystalline modification A which has an X-ray diffraction peak at 10.69±0.20 (2Θ).

3. The crystalline modification A according to claim 2, wherein said crystalline modification further has at least one X-ray diffraction peak selected from the group consisting of 12.81±0.20 (2Θ), 13.82±0.20 (2Θ), 13.88±0.20 (2Θ), 16.71±0.20 (2Θ), 18.31±0.20 (2Θ), 18.76±0.20 (2Θ), 19.52±0.20 (2Θ), 20.56±0.20 (2Θ), 20.60±0.20 (2Θ), 20.61±0.20 (2Θ), 21.42±0.20 (2Θ), 22.63±0.20 (2Θ), 23.85±0.20 (2Θ) and 26.34±0.20 (2Θ).

4. The crystalline modification A according to claim 3, wherein said crystalline modification additionally has at least one X-ray diffraction peak selected from the group consisting of 18.32±0.20 (2Θ), 24.79±0.20 (2Θ), 25.08±0.20 (2Θ), 28.66±0.20 (2Θ), 30.33±0.20 (2Θ), 33.05±0.20 (2Θ) and 38.36±0.20 (2Θ).

5. The crystalline modification A according to claim 4, wherein said crystalline modification additionally has at least one X-ray diffraction peak selected from the group consisting of 10.24±0.20 (2Θ), 10.77±0.20 (2Θ), 14.22±0.20 (2Θ), 17.52±0.20 (2Θ), 19.89±0.20 (2Θ), 21.48±0.20 (2Θ), 21.64±0.20 (2Θ), 23.22±0.20 (2Θ), 23.37±0.20 (2Θ), 25.67±0.20 (2Θ), 25.77±0.20 (2Θ), 26.33±0.20 (2Θ), 27.85±0.20 (2Θ), 28.59±0.20 (2Θ), 28.82±0.20 (2Θ), 29.43±0.20 (2Θ), 29.67±0.20 (2Θ), 29.93±0.20 (2Θ), 30.11±0.20 (2Θ), 30.17±0.20 (2Θ), 30.52±0.20 (2Θ), 31.62±0.20 (2Θ), 32.31±0.20 (2Θ), 32.46±0.20 (2Θ), 32.59±0.20 (2Θ), 32.71±0.20 (2Θ), 33.67±0.20 (2Θ), 33.71±0.20 (2Θ), 33.79±0.20 (2Θ), 33.92±0.20 (2Θ), 33.92±0.20 (2Θ), 34.23±0.20 (2Θ), 34.28±0.20 (2Θ), 34.39±0.20 (2Θ), 35.02±0.20 (2Θ), 35.24±0.20 (2Θ), 35.46±0.20 (2Θ), 35.61±0.20 (2Θ), 35.80±0.20 (2Θ), 36.53±0.20 (2Θ), 36.75±0.20 (2Θ), 36.92±0.20 (2Θ), 37.14±0.20 (2Θ), 37.16±0.20 (2Θ), 37.34±0.20 (2Θ), 38.08±0.20 (2Θ), 38.38±0.20 (2Θ), 38.45±0.20 (2Θ), 38.82±0.20 (2Θ), 39.29±0.20 (2Θ), 39.36±0.20 (2Θ), 39.47±0.20 (2Θ) and 39.63±0.20 (2Θ).

6. The crystalline modification A according to claim 2, wherein in DSC analyses said crystalline modification exhibits an endothermal event with a peak temperature in the range of 113-121° C.

7. The crystalline modification A according to claim 6, wherein in DSC analyses said crystalline modification exhibits an endothermal event with a peak temperature in the range of 114-120° C.

8. The crystalline modification A according to claim 7, wherein in DSC analyses said crystalline modification exhibits an endothermal event with a peak temperature in the range of 115-119° C.

9. The crystalline modification A according to claim 8, wherein in DSC analyses said crystalline modification exhibits an endothermal event with a peak temperature in the range of 115-118° C.

10. A crystalline modification according to claim 1, wherein said crystalline modification is crystalline modification B which has at least one X-ray diffraction peak selected from the group consisting of 11.35±0.20 (2Θ) and 24.30±0.20 (2Θ).

11. The crystalline modification B according to claim 10, wherein said crystalline modification additionally has at least one X-ray diffraction peak selected from the group consisting of 12.75±0.20 (2Θ), 14.04±0.20 (2Θ), 16.51±0.20 (2Θ), 18.79±0.20 (2Θ), 19.74±0.20 (2Θ), 20.09±0.20 (2Θ) and 21.20±0.20 (2Θ).

12. The crystalline modification B according to claim 11, wherein said crystalline modification additionally has at least one X-ray diffraction peak selected from the group consisting of 15.23±0.20 (2Θ), 19.20±0.20 (2Θ), 21.42±0.20 (2Θ), 23.69±0.20 (2Θ), 23.76±0.20 (2Θ), 24.30±0.20 (2Θ), 25.66±0.20 (2Θ), 25.74±0.20 (2Θ), 25.84±0.20 (2Θ), 28.30±0.20 (2Θ) and 31.81±0.20 (2Θ).

13. The crystalline modification B according to claim 12, wherein said crystalline modification additionally has at least one X-ray diffraction peak selected from the group consisting of 9.72±0.20 (2Θ), 12.79±0.20 (2Θ), 22.82±0.20 (2Θ), 26.55±0.20 (2Θ), 26.77±0.20 (2Θ), 27.07±0.20 (2Θ), 27.83±0.20 (2Θ), 28.07±0.20 (2Θ), 28.49±0.20 (2Θ), 29.44±0.20 (2Θ), 29.74±0.20 (2Θ), 30.21±0.20 (2Θ), 30.27±0.20 (2Θ), 30.62±0.20 (2Θ), 30.74±0.20 (2Θ), 31.96±0.20 (2Θ), 32.01±0.20 (2Θ), 32.09±0.20 (2Θ), 33.18±0.20 (2Θ), 33.39±0.20 (2Θ), 33.94±0.20 (2Θ), 34.01±0.20 (2Θ), 34.25±0.20 (2Θ), 34.52±0.20 (2Θ), 34.85±0.20 (2Θ), 35.37±0.20 (2Θ), 35.55±0.20 (2Θ), 35.70±0.20 (2Θ), 35.83±0.20 (2Θ), 37.21±0.20 (2Θ), 37.29±0.20 (2Θ), 37.63±0.20 (2Θ), 38.12±0.20 (2Θ), 38.20±0.20 (2Θ), 38.48±0.20 (2Θ), 38.50±0.20 (2Θ), 38.96±0.20 (2Θ), 39.04±0.20 (2Θ), 39.47±0.20 (2Θ) and 39.97±0.20 (2Θ).

14. The crystalline modification B according to claim 10, wherein in DSC analyses said crystalline modification exhibits an endothermal event with a peak temperature in the range of 109-120° C.

15. The crystalline modification B according to claim 14, wherein in DSC analyses said crystalline modification exhibits an endothermal event with a peak temperature in the range of 110-119° C.

16. The crystalline modification B according to claim 15, wherein in DSC analyses said crystalline modification exhibits an endothermal event with a peak temperature in the range of 111-118° C.

17. The crystalline modification B according to claim 16, wherein in DSC analyses said crystalline modification exhibits an endothermal event with a peak temperature in the range of 112-115° C.

18. A crystalline modification according to claim 1, wherein said crystalline modification is crystalline modification C which has at least one X-ray diffraction peak selected from the group consisting of 9.05±0.20 (2Θ), 14.64±0.20 (2Θ), 15.83±0.20 (2Θ) and 16.07±0.20 (2Θ).

19. The crystalline modification C according to claim 18, wherein said crystalline modification additionally has at least one X-ray diffraction peak selected from the group consisting of 15.47±0.20 (2Θ), 16.84±0.20 (2Θ), 18.07±0.20 (2Θ), 19.64±0.20 (2Θ), 20.23±0.20 (2Θ), 21.04±0.20 (2Θ), 21.49±0.20 (2Θ), 22.04±0.20 (2Θ), 24.79±0.20 (2Θ), 25.69±0.20 (2Θ), 27.80±0.20 (2Θ), 28.22±0.20 (2Θ) and 31.17±0.20 (2Θ).

20. The crystalline modification C according to claim 19, wherein said crystalline modification additionally has at least one X-ray diffraction peak selected from the group consisting of 12.54±0.20 (2Θ), 15.02±0.20 (2Θ), 17.77±0.20 (2Θ), 24.94±0.20 (2Θ), 25.18±0.20 (2Θ), 25.82±0.20 (2Θ), 26.34±0.20 (2Θ), 26.82±0.20 (2Θ), 29.25±0.20 (2Θ), 29.46±0.20 (2Θ), 29.89±0.20 (2Θ), 30.07±0.20 (2Θ), 34.00±0.20 (2Θ), 35.90±0.20 (2Θ), 36.34±0.20 (2Θ) and 39.12±0.20 (2Θ).

21. The crystalline modification C according to claim 20, wherein said crystalline modification additionally has at least one X-ray diffraction peak selected from the group consisting of 10.04±0.20 (2Θ), 23.78±0.20 (2Θ), 30.31±0.20 (2Θ), 30.64±0.20 (2Θ), 32.47±0.20 (2Θ), 32.94±0.20 (2Θ), 33.21±0.20 (2Θ), 34.40±0.20 (2Θ), 38.13±0.20 (2Θ) and 39.31±0.20 (2Θ).

22. The crystalline modification C according to claim 18, wherein in DSC analyses said crystalline modification exhibits an endothermal event with a peak temperature in the range of 113-124° C.

23. The crystalline modification C according to claim 22, wherein in DSC analyses said crystalline modification exhibits an endothermal event with a peak temperature in the range of 114-123° C.

24. The crystalline modification C according to claim 23, wherein in DSC analyses said crystalline modification exhibits an endothermal event with a peak temperature in the range of 115-122° C.

25. The crystalline modification C according to claim 24, wherein in DSC analyses said crystalline modification exhibits an endothermal event with a peak temperature in the range of 116-121° C.

26. A pharmaceutical composition comprising at least one crystalline modification of (1R,3R,6R)-6-dimethylaminomethyl-1-(3-methoxyphenyl)-cyclohexane-1,3-diol, or (1S,3S,6S)-6-dimethylaminomethyl-1-(3-methoxyphenyl)-cyclohexane-1,3-diol, or a mixture thereof and at least one pharmaceutically acceptable carrier, additive or adjuvant.

27. A pharmaceutical composition according to claim 26, wherein said crystalline modification is crystalline modification A.

28. A pharmaceutical composition according to claim 26, wherein said crystalline modification is crystalline modification B.

29. A pharmaceutical composition according to claim 26, wherein said crystalline modification is crystalline modification C.

30. A process for obtaining crystalline (1R,3R,6R)-6-dimethylaminomethyl-1-(3-methoxyphenyl)-cyclohexane-1,3-diol or (1S,3S,6S)-6-dimethylaminomethyl-1-(3-methoxyphenyl)-cyclohexane-1,3-diol, said process comprising cooling a solution of a mixture of the enantiomers thereof.
